(19) United States
(12) Patent Application Publication
(10) Pub. No.: US 2025/0270618 A1
(43) Pub. Date: Aug. 28, 2025

(54) METHODS FOR DIAGNOSIS AND TREATMENT OF ALZHEIMER'S DISEASE

(71) Applicant: The Children's Medical Center Corporation, Boston, MA (US)

(72) Inventors: Hanno STEEN, Brighton, MA (US); Judith STEEN, Brighton, MA (US); Patrick W. VAN ZALM, Boston, MA (US)

(73) Assignee: The Children's Medical Center Corporation, Boston, MA (US)

(21) Appl. No.: 18/549,676

(22) PCT Filed: Feb. 28, 2022
(86) PCT No.: PCT/US2022/018087

§ 371 Date: Sep. 8, 2023

Related U.S. Application Data
(60) Provisional application No. 63/157,896, filed on Mar. 8, 2021.

(51) Int. Cl. C12Q 1/48; A61K 31/13; A61K 31/27; A61K 31/445; A61K 31/55; C07K 16/18; C12Q 1/32; C12Q 1/527; G01N 33/68; G01N 33/92

(52) U.S. Cl. CPC C12Q 1/485; A61K 31/13; A61K 31/27; A61K 31/445; A61K 31/55; C07K 16/18; C12Q 1/32; C12Q 1/527; G01N 33/6896; G01N 33/92; G01N 2333/4703; G01N 2333/775; G01N 2333/902; G01N 2333/91215; G01N 2333/988; G01N 2800/2821; G01N 2800/52

(57) ABSTRACT

Provided herein are methods for diagnosing and treating Alzheimer's disease in a subject comprising determining the expression level of three, four or five members of a panel of proteins in a biological sample obtained from the subject.

### CROSS-REFERENCE TO RELATED APPLICATIONS

This application is a 35 U.S.C. § 371 National Phase Entry Application of International Application No. PCT/US2022/018087 filed Feb. 28, 2022, which designates the U.S. and claims benefit under 35 U.S.C. § 119(e) of U.S. Provisional Application No. 63/157,896 filed Mar. 8, 2021, the contents each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Mar. 30, 2022, is named 701039-099180WOPT_SL.txt and is 2,456 bytes in size.

### FIELD OF THE INVENTION

The field of the invention relates to the diagnosis and treatment of Alzheimer's disease or a symptom thereof.

### BACKGROUND

Alzheimer's disease (AD) is a form of dementia that occurs in aging adults. AD gives rise to an irreversible progressive loss of cognitive functions, which can include severe memory impairment, confusion, language disturbances, impaired judgment, personality and behavioral changes. During advanced stages of the disease, patients are often unable to care for themselves and require institutionalization or professional care in the home setting. While some patients may live for many years after being diagnosed with AD, the average life expectancy after diagnosis is eight years.

The incidence of AD is projected to increase dramatically over the next few decades and there are few treatments available for use. Such treatments have limited efficacy for treatment of Alzheimer's disease itself and serve mostly to slow or delay progression of dementia symptoms.

### SUMMARY

The compositions and methods described herein are based, in part, on the discovery of a panel of biomarker proteins that can be detected non-invasively in cerebrospinal fluid (CSF) or blood, which can serve to diagnose and inform treatment of Alzheimer's disease in a subject. The panel of biomarker proteins comprises at least two of the following proteins: pyruvate kinase (PKM), fructose bisphosphate aldolase A (ALDOA), lactate dehydrogenase B chain (LDHB), 14-3-3 protein zeta/delta (YWHAZ), and apolipoprotein E isoform 4 (apoE4).

Accordingly, provided herein in one aspect is a method for treating Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), and lactate dehydrogenase B chain (LDHB), (ii) determining that the expression of at least one of the members of the panel of proteins is increased in the biological sample as compared to the expression of the at least one member in a reference sample, and (iii) administering a treatment for Alzheimer's disease to the subject, thereby treating Alzheimer's disease in the subject.

Another aspect provided herein relates to a method for reducing at least one symptom of Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), and lactate dehydrogenase B chain (LDHB), (ii) determining that the expression of at least one of the members of the panel of proteins is increased in the biological sample as compared to the expression of the at least one member in a reference sample, thereby indicating that the subject has Alzheimer's disease and (iii) administering a treatment for Alzheimer's disease to the subject, wherein the treatment reduces at least one symptom of the Alzheimer's disease.

In one embodiment of this aspect and all other aspects provided herein, the biological sample is cerebrospinal fluid (CSF), whole blood, plasma, serum, or urine. In another embodiment of this aspect and all other aspects provided herein, the biological sample comprises exosomes.

In another embodiment of this aspect and all other aspects provided herein, the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

In another embodiment of this aspect and all other aspects provided herein, the method further comprises a step of obtaining the biological sample from the subject.

In another embodiment of this aspect and all other aspects provided herein, the method further comprises a step of selecting the subject based on the presence or expression level of apolipoprotein E isoform 4 (apoE4).

In another embodiment of this aspect and all other aspects provided herein, the treatment for Alzheimer's disease comprises a cholinesterase inhibitor, intravenous immunoglobulin (IVIG), aducanumab, or memantine.

In another embodiment of this aspect and all other aspects provided herein, the cholinesterase inhibitor comprises donepezil, galantamine, or rivastigmine.

In another embodiment of this aspect and all other aspects provided herein, the method further comprises repeating steps (i) and (ii) at a later time point after treatment has been initiated to monitor efficacy of the treatment.

Another aspect provided herein is a method for treating Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), and 14-3-3 protein zeta/delta (YWHAZ), (ii) determining that the expression of at least one of the members of the panel of proteins is increased in the biological sample as compared to the expression of the at least one member in a reference sample, (iii) administering a treatment for Alzheimer's disease to the subject, thereby treating Alzheimer's disease in the subject.

Another aspect provided herein is a method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), and 14-3-3 protein zeta/delta (YWHAZ), (ii) determining that the expression of at least one of the members of the panel of proteins is increased in the biological sample as compared to the expression of the at least one member in a reference sample, and (iii) administering a treatment for Alzheimer's disease to the subject, wherein the treatment reduces or alleviates one or more symptoms of Alzheimer's disease in the subject.

In one embodiment of these aspects and all other aspects provided herein, the biological sample is cerebrospinal fluid (CSF), whole blood, plasma, serum, or urine. In another embodiment of this aspect and all other aspects provided herein, the biological sample comprises exosomes.

In another embodiment of this aspect and all other aspects provided herein, the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

In another embodiment of this aspect and all other aspects provided herein, the method further comprises a step of obtaining the biological sample from the subject.

In another embodiment of this aspect and all other aspects provided herein, the method further comprises a step of selecting the subject based on the presence or expression level of apolipoprotein E isoform 4 (apoE4).

In another embodiment of this aspect and all other aspects provided herein, the treatment for Alzheimer's disease comprises a cholinesterase inhibitor, intravenous immunoglobulin (IVIG), aducanumab, or memantine.

In another embodiment of this aspect and all other aspects provided herein, the cholinesterase inhibitor comprises donepezil, galantamine, or rivastigmine.

In another embodiment of this aspect and all other aspects provided herein, the method further comprises repeating steps (i) and (ii) at a later time point after treatment has been initiated to monitor efficacy of the treatment.

Another aspect provided herein relates to a method for treating Alzheimer's disease in a subject, the method comprising: (i)) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), and apolipoprotein E isoform 4 (apoE4), (ii) determining that the expression of at least one of the members of the panel of proteins is increased in the biological sample as compared to the expression of the at least one member in a reference sample, (iii) administering a treatment for Alzheimer's disease to the subject, thereby treating Alzheimer's disease in the subject.

Another aspect provided herein relates to a method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), and apolipoprotein E isoform 4 (apoE4), (ii) determining that the expression of at least one of the members of the panel of proteins is increased in the biological sample as compared to the expression of the at least one member in a reference sample, (iii) administering a treatment for Alzheimer's disease to the subject, wherein the treatment for Alzheimer's disease reduces or alleviates one or more symptoms in the subject.

In one embodiment of this aspect and all other aspects provided herein, the biological sample is cerebrospinal fluid (CSF), whole blood, plasma, serum or urine. In another embodiment of this aspect and all other aspects provided herein, the biological sample comprises exosomes.

In another embodiment of this aspect and all other aspects provided herein, the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

In another embodiment of this aspect and all other aspects provided herein, the method further comprises a step of obtaining the biological sample from the subject.

In another embodiment of this aspect and all other aspects provided herein, the treatment for Alzheimer's disease comprises a cholinesterase inhibitor, intravenous immunoglobulin (IVIG), aducanumab, or memantine.

In another embodiment of this aspect and all other aspects provided herein, the cholinesterase inhibitor comprises donepezil, galantamine, or rivastigmine.

In another embodiment of this aspect and all other aspects provided herein, the method further comprises repeating steps (i) and (ii) at a later time point after treatment has been initiated to monitor efficacy of the treatment.

A method for treating Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, (iii) administering a treatment for Alzheimer's disease to the subject, thereby treating Alzheimer's disease in the subject.

A method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises (or consists essentially of) pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, (iii) administering a treatment for Alzheimer's disease to the subject, thereby treating Alzheimer's disease in the subject.

In one embodiment of this aspect and all other aspects provided herein, the biological sample is cerebrospinal fluid (CSF), whole blood, plasma, serum, or urine. In another embodiment of this aspect and all other aspects provided herein, the biological sample comprises exosomes.

In another embodiment of this aspect and all other aspects provided herein, the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

In another embodiment of this aspect and all other aspects provided herein, the method further comprises a step of obtaining the biological sample from the subject.

In another embodiment of this aspect and all other aspects provided herein, the treatment for Alzheimer's disease comprises a cholinesterase inhibitor, intravenous immunoglobulin (IVIG), aducanumab, or memantine.

In another embodiment of this aspect and all other aspects provided herein, the cholinesterase inhibitor comprises donepezil, galantamine, or rivastigmine.

In another embodiment of this aspect and all other aspects provided herein, the method further comprises repeating steps (i) and (ii) at a later time point after treatment has been initiated to monitor efficacy of the treatment.

Another aspect provided herein relates to a method for diagnosing Alzheimer's disease in a subject, the method comprising: (i) receiving results of a protein assay relating to the expression of each member of a panel of proteins in a biological sample obtained from a subject, wherein the panel of proteins comprises at least two of the members of, or consists essentially of, pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, thereby diagnosing the subject as having Alzheimer's Disease.

Another aspect provided herein relates to a method for selecting a subject to be treated for Alzheimer's disease, the method comprising: (i) receiving results of a protein assay relating to the expression of each member of a panel of proteins in a biological sample obtained from a subject, wherein the panel of proteins comprises at least two of the members of, or consists essentially of, pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, thereby selecting the subject to be treated for Alzheimer's Disease.

In one embodiment of this aspect and all other aspects provided herein, the subject is selected for treatment for Alzheimer's disease by participation in a clinical trial for a therapeutic to treat Alzheimer's disease. Such therapeutics can be early-stage interventions or anti-Tau therapies to prevent or slow progression of the disease.

Another aspect provided herein relates to a method for reducing or alleviating one or more symptoms of Alzheimer's disease in a subject, the method comprising: (i) receiving results of an mRNA assay relating to the expression of each member of a panel of gene products in a biological sample obtained from a subject, wherein the panel of gene products comprises (or consists essentially of) the members in panels 1, 2, 3, or 4 as described herein in Table 1, (ii) determining that the expression of at least one of the members of the panel of gene products is increased in the biological sample as compared to the expression of the at least one member in a reference sample, (iii) administering a treatment for Alzheimer's disease to the subject, wherein the treatment for Alzheimer's disease reduces or alleviates one or more symptoms in the subject.

In one embodiment of this aspect and all other aspects provided herein, the biological sample comprises circulating RNA. In another embodiment of this aspect and all other aspects provided herein, the biological sample comprises exosomes.

### BRIEF DESCRIPTION OF THE FIGURES

FIGS. 1A-1F Data collection and pre-processing. The literature was searched on PubMed, resulting in 394 records of which 6 did result in the collection of raw MS data after exclusion and inclusion criteria were taken into account (FIG. 1A). With the addition of one in-house dataset a total of 150 samples were collections, derived from 7 cohorts across 5 different countries. All data was systematically re-analyzed in MaxQuant™ (FIG. 1B) followed by data normalization (FIG. 1C). For the systematic removal of outliers, a method was developed where a theoretical sample was created by calculating the median intensity of each protein in a dataset (FIG. 1D). Next, Pearson correlation between each of the individual samples and the theoretical sample was calculated and any sample that was more than three standard deviations removed from the theoretical sample were considered outliers and removed from downstream analysis. The process described in FIGS. 1B, 1C and 1D was repeated for each of the 7 cohorts whereafter data was combined resulting in a PCA plot as shown in FIG. 1E. To overcome the variability between datasets a Z-score transformation was used, which resulted in a homogenous dataset (FIG. 1F). Z-score transformation was considered an easy and effective method to make heterogeneous data comparable with one another for downstream analysis.

FIGS. 2A-2E. Statistical and bioinformatic analysis. The Z-scored data was statistically analyzed with the Mann-Whitney U test and Benjamini-Hochberg multiple comparison correction, leading to 15 significant proteins (FIG. 2A). In comparison, many of these 15 proteins were not found to be significant in individual datasets, or datasets did contradict one another (FIG. 2B). The procedural removal of one cohort followed by statistical analysis did result in an additional 20 biomarker candidates. The resulting 35 biomarker candidates were analyzed in STRING, resulting in a strongly connected protein-protein network (FIG. 2C). A total of 166 GO biological process annotations were associated with this protein-protein network, of which the top 10 (FDR) are shown (FIG. 2D). Next, the ClueGO software was used to group and visualize the associated GO annotations. ClueGO did result in three clusters of GO annotations, of which the cluster associated with metabolic processes was the largest (FIG. 2E). All proteins, biological process and ClueGO clusters that were linked to metabolic processes are highlighted, indicating that the discovered biomarker candidates play crucial roles in glucose homeostasis.

FIGS. 3A-3B. Biomarker validation and biomarker panel development. Statistical analysis (Mann-Whitney U test and Benjamini-Hochberg correction) of the validation cohort did result in 24 significant proteins (FIG. 3A). Furthermore, the 4 validated biomarker candidates ALDOA, PKM, LDHB and APOE-4 showed high AUROCs (>0.80) in the validation cohort. Next, these 4 validated biomarker candidates were used to develop a biomarker panel. For the development of the biomarker panel, the discovery dataset was used to train a logistic regression model, which was validated in our validation cohort as well as two recently published large CSF proteomics studies (FIG. 3B). APOE-4 was not observed in the two large proteomics studies and therefore ALDOA, PKM and LDHB were used for the biomarker panel development. The developed panel was effective in differentiating AD from controls throughout three validation cohorts. This biomarker panel which is made up of three glycolytic enzymes can therefore be an excellent representation of glucose homeostasis pathology in AD.

### DETAILED DESCRIPTION

Provided herein are compositions and methods for diagnosing and treating Alzheimer's disease. In particular, provide herein are methods for diagnosing a subject as having AD and informing their treatment by analyzing a biological sample for the expression level of each member of a panel of proteins. Exemplary protein panels include (i) pyruvate kinase (PKM), fructose bisphosphate aldolase A (ALDOA) and lactate dehydrogenase B chain (LDHB); (ii) PKM, ALDOA, LDHB and 14-3-3 protein zeta/delta (YWHAZ); (iii) PKM, ALDOA, LDHB and apolipoprotein E isoform 4 (apoE4); and (iv) PKM, ALDOA, LDHB, YWHAZ, and apoE4.

### Definitions

The terms “patient,” “subject” and “individual” are used interchangeably herein, and refer to an animal, particularly a human, to whom treatment, including prophylactic treatment is provided.

As used herein, a “reference value” of a given biomarker or member of a panel of proteins can be an absolute value, a relative value, a value that has an upper and/or lower limit, a range of values, an average value, a median value, a mean value, a value as compared to a particular control or baseline value or a combination thereof. It is to be understood that other statistical variables can be used in determining the reference value. A reference value can be based on an individual sample value; for example, a value obtained from a sample from the same individual having Alzheimer's disease, but at an earlier point in time, or a value obtained from a sample from an Alzheimer's disease patient other than the individual being tested or a population thereof, or a “normal” individual, that is an individual not diagnosed with Alzheimer's disease (AD) or a population thereof. The reference value can be based on a large number of samples, such as from a population of AD patients or normal individuals or based on a pool of samples including or excluding the sample to be tested.

As used herein, “biomarker panel” refers to a set of biomarkers that can be used together, for example, in combinations or sub-combinations for the detection, diagnosis, prognosis, staging, or monitoring of a disease or condition, based on detection values for the set of biomarkers. The panel of biomarkers described herein comprises pyruvate kinase (PKM), fructose bisphosphate aldolase A (ALDOA), lactate dehydrogenase B chain (LDHB), 14-3-3 protein zeta/delta (YWHAZ), and apolipoprotein E isoform 4 (apoE4). Exemplary combinations or sub-combinations of these biomarkers include: (i) PKM, ALDOA and LDHB; (ii) PKM, ALDOA, LDHB and YWHAZ; (iii) PKM, ALDOA, LDHB and apoE4; and (iv) PKM, ALDOA, LDHB, YWHAZ, and apoE4.

The terms “decrease”, “reduced”, “reduction”, or “inhibit” are all used herein to mean a decrease or lessening of a property, level, or other parameter (such as a biological marker or a disease symptom) by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease in a symptom of Alzheimer's disease can be preferably down to a level accepted as within the range of normal for an individual without Alzheimer's disease.

The terms “increased,” “increase” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. In one embodiment, a reference level can be the amount of each member of the panel of proteins in a subject or population of subjects lacking Alzheimer's disease.

The term “pharmaceutically acceptable” can refer to compounds and compositions which can be administered to a subject (e.g., a human) without undue toxicity.

As used herein, the term “pharmaceutically acceptable carrier” can include any material or substance that, when combined with an active ingredient, allows the ingredient to retain biological activity and is substantially non-reactive with the subject's immune system (unless desired). Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture and bacterial culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### Alzheimer's Disease and Conventional Diagnosis Thereof

Alzheimer's Disease (AD) is the most common form of dementia that makes up 60 to 80% of all dementia cases (Erkkinen, Kim, & Geschwind, 2018). It has been estimated that about 50 million patients suffer from AD worldwide and that number is expected to increase to 130 million in 2050 (Alzheimer's Association, 2017). While the number of patients is increasing, no successful treatment has been discovered yet. One major hurdle in the development of treatments for AD is differentiation AD patients from patients with other dementias. So far, the only way to definitively differentiate AD is by post mortem brain examination. The lack of robust in vivo diagnostics underscores the urgent need for such diagnostic modality.

In AD, the best known and most researched physio-pathological abnormality found in the brain is the accumulation of the amyloid-beta A4 protein (APP or A4_human) into β-amyloid (Aβ) plaques (Brier et al., 2016). The accumulation of Aβ plaques is primarily observed in the extracellular space of neurons caused by the miscleavage of APP (Blennow, Hampel, Weiner, & Zetterberg, 2010). Under normal conditions, APP is cleaved by α-secretase into soluble Amyloid Precursor Protein a but in pathological conditions APP is miscleaved by β-secretase leading to soluble Amyloid Precursor Protein f which has two main isoforms: Aβ1-42 (Aβ42) and AβD1-40 (AβP40) (Gu & Guo, 2013). Subsequently, Aβ42 aggregates into Aβ42 plaques in the extracellular space of neurons in the cerebral cortex which has been observed up to ten years before the symptomatic onset of AD (Buchhave et al., 2012).

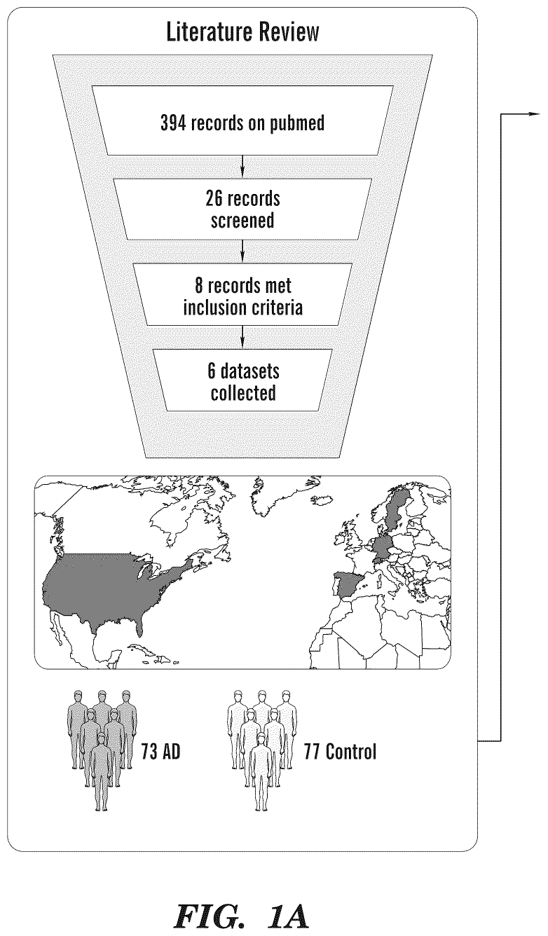

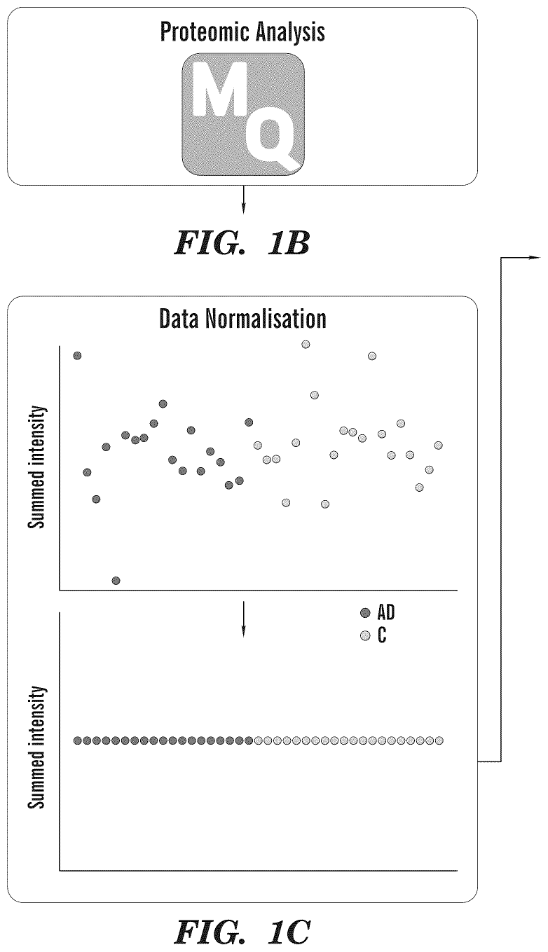

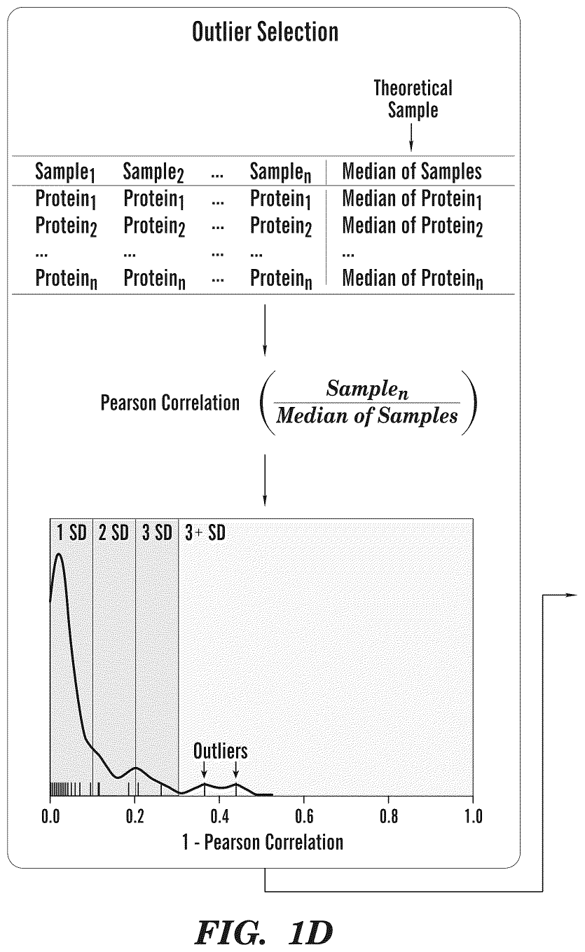

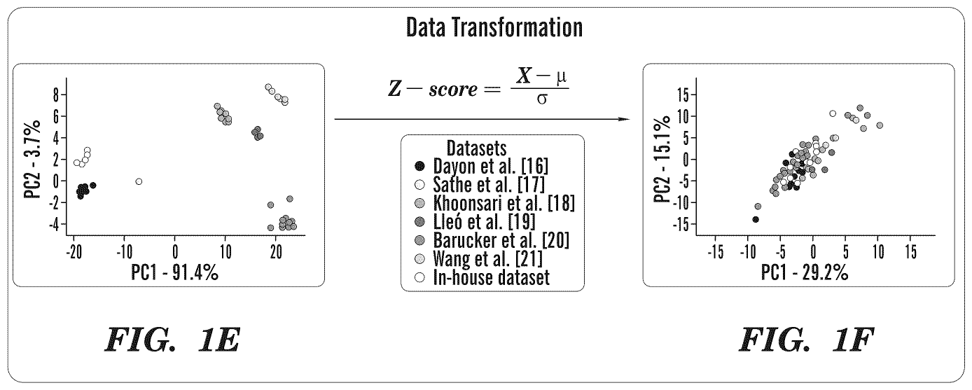

Another extensively researched physio-pathological abnormality in AD is the hyper-phosphorylation of the protein tau (Brier et al., 2016). The protein tau is translated from the microtubule-associated protein tau gene (MAPT or TAU_human), which comprises 16 exons on 17q21 (Y. Wang & Mandelkow, 2016). In the human adult brain, alternative splicing of tau gives rise to six major isoforms (FIG. 1) which are generated by the alternative splicing of exons 2, 3 and 10. The function of tau is to promote the assembly of microtubules that stabilize the structure of neurons (Iqbal, Liu, Gong, & Grundke-Iqbal, 2010). The process of microtubule stabilization is driven through post-translational modification (PTM) of tau with the most prominent type of PTM being phosphorylation. In pathological conditions, the phosphorylation of tau is increased resulting in hyper-phosphorylated tau (p-tau) which in turn leads to the detachment of tau from the microtubules causing their disassembly. Also, the detached tau will aggregate forming paired helical filaments (PHF) which consequently lead to the aggregation of the PHF's into intracellular neurofibrillary tangles (NFT) (Medeiros, Baglietto-Vargas, & LaFerla, 2011). The disassembly of the microtubules and the formation of intracellular NFTs cause disruption of the functioning of the neuron which ultimately can lead to neuronal death. Other diseases share the pathological conditions of the aggregation of tau and these diseases have therefore been called tauopathies (Irwin, 2016). Tauopathies have been divided in two types: primary and secondary tauopathies (Lebouvier, Pasquier, & Bude, 2017). This distinction was created to differentiate diseases that are characterized by tau aggregates as the main pathology (primary) from diseases that show tau aggregates as one of multiple pathologies (secondary), such as AD.

Clinically, the symptoms of AD manifest slowly and the initial signs may only be mild forgetfulness. In this early stage, individuals have a tendency to forget recent events, activities, the names of familiar people or things and may not be able to solve simple mathematical problems. As the disease progresses into moderate stages of AD, symptoms are more easily detected and become serious enough to cause people with AD or their family members to seek medical help. Moderate stage symptoms of AD include the inability to perform simple tasks such as grooming, and problems in speech, understanding, reading, and writing. Severe stage AD patients may become anxious or aggressive, may wander away from home, and ultimately will need total care.

Alzheimer's disease is primarily diagnosed by exclusion of other known causes of dementia as this disease can only be definitively confirmed post-mortem. Early stage Alzheimer's detection would be beneficial as it would permit therapeutic intervention to be administered prior to onset of irreversible changes to the brain.

Neuroimaging and neurocognitive tests are two cornerstones of the current medical practice used to diagnose Alzheimer's disease. Neurocognitive tests are easier to implement as compared to neuroimaging, however the results should be evaluated with care as these tests are easily influenced by other factors such as education, cultural background, social economic status, etc. Hence, neurocognitive test results should not be the only information to reach a final diagnosis of Alzheimer's disease. As for neuroimaging, structural or functional data can be obtained to diagnose Alzheimer's disease on an objective ground. For example, hippocampal atrophy can be identified either qualitatively (visual rating) or quantitatively (volumetry) by using magnetic resonance imaging; and amyloid or tau positron emission tomography (PET) can show amyloid plaques and neurofibrillary tangles typically found in the brains of Alzheimer's disease patients. Neuroimaging is not always accessible, particularly in private clinics or small hospitals.

Methods for clinical diagnosis and staging of Alzheimer's disease are known to those of skill in the art, and include, but are not limited to Mini-Mental State Examination (MMSE, Folstein 1975), Diagnostic and Statistical Manual of Mental Disorders (DSM)-IV criteria (American Psychiatric Association: DSM-IV: Diagnostic and Statistical Manual of Mental Disorders, Washington DC: American Psychiatric Association (1994)) criteria, NINCDS-ADRDA criteria (McKhann G et al. Neurology (1984) 34: 939-944)), or the Clinical Dementia Rating Scale (CDR) (Hughes, C. et al (1982) British Journal of Psychiatry, 140, 566-572) or a combination thereof. For example, using the MMSE, scores of 27 or above (out of 30) are considered normal. An individual scoring between 21 and 26 points is viewed as having mild AD. An individual scoring between 10 and 20 points is viewed as having moderate AD and less than 10 points is rated as severe AD. On average people with Alzheimer's disease who do not receive treatment lose 2 to 4 points each year on the MMSE scoring system.

As used herein, a subject lacking Alzheimer's disease or another form of dementia are referred to herein as “normal” or “a non-Alzheimer's subject.” Such subjects will typically have a value of zero on the clinical dementia rating scale (CRS) established by McKhann et al. (McKhann G, et al. Neurology. 1984; 34: 939-944).

### Biomarker Panels

Provided herein are biomarkers that can be used in a panel for the diagnosis of Alzheimer's disease or to inform treatment of AD especially during early-stage disease. The biomarkers for use in a panel include pyruvate kinase (PKM; e.g., NCBI Accession No. P14618.4), fructose bisphosphate aldolase A (ALDOA; e.g., NCBI Accession No. P04075.2), lactate dehydrogenase B chain (LDHB; e.g., NCBI Accession No. P07195.2), 14-3-3 protein zeta/delta (YWHAZ e.g., NCBI Accession No. P63104.1), and apolipoprotein E isoform 4 (apoE4; e.g., NCBI Accession No. P02649.1). Typically, an assay as described herein will comprise detection or quantification of at least two of the biomarkers in the panel. In other embodiments, the assays described herein comprise detection or quantification of at least three, at least four, or at all 5 of these biomarkers in the panel.

While any combination of these five biomarkers is contemplated for use in a biomarker panel for use as described herein, exemplary biomarker panels include those shown in the Table 1, as follows:

| | Biomarkers |
|---|---|
| Panel 1 | PKM, ALDOA, LDHB |
| Panel 2 | PKM, ALDOA, LDHB, YWHAZ |
| Panel 3 | PKM, ALDOA, LDHB, apoE4 |
| Panel 4 | PKM, ALDOA, LDHB, YWHAZ, |
| | apoE4 |

An exemplary peptide sequence for each of the biomarkers in the panel is shown in Table 2, as follows:

| | | SEQ |
|---|---|---|
| Biomarker | Protein Sequence | ID NO: |
| PKM | LNFSHGTHEYHAETIKTATESFASDPILYRPVAVALDTKGPEIRSVETLKEMIK | 1 |
| ALDOA | LQSIGTENTEENRRADDGRPFPQVIKIGEHTPSALAIMENANVLAR | 2 |
| LDHB | IVADKDYSVTANSKIVVVTAGVRLKDDEVAQLKK | 3 |
| YWHAZ | NLLSVAYKMDKNELVQKSVTEQGAELSNEER | 4 |
| apoE4 | LGADMEDVCGRLGADMEDVRLAVYQAGARCLAVYQAGARLGPLVEQGR | 5 |

### Biological Samples

It is specifically contemplated herein that any biological sample can be used to assess the expression level or amount of biomarkers in the biomarker panel described herein. However, biological samples that can be obtained non-invasively or less-invasively are preferred over other biological samples. Non-limiting examples of biological samples include, but not limited to, cerebrospinal fluid (CSF), blood, serum, plasma, urine, saliva, circulating cells, circulating RNA, exosomes, peritoneal fluid, lymph fluid, interstitial fluid, tissue homogenate, cell extracts, stool, and extracts of tissues including biopsies of tissues or any other constituents of the body which may contain the biomarkers of interest.

In one embodiment, the biological sample comprises CSF.

In another embodiment, the biological sample comprises whole blood or a portion thereof (e.g., serum, plasma, circulating cells, circulating RNA etc).

### Biomarker Assays

The biomarkers or combination thereof used herein to predict, diagnose, or monitor AD can be measured using any process known to those of skill in the art including, but not limited to, enzyme linked immunosorbent assay (ELISA), fluorescence polarization immunoassay (FPIA) and homogeneous immunoassays, point of care tests using conventional lateral flow immunochromatography (LFA), quantitative point of care tests using determination of chemiluminescence, fluorescence, and magnetic particles, as well as latex agglutination, biosensors, gel electrophoresis, mass spectrometry (MS), gas chromatograph-mass spectrometry (GC-MS), nanotechnology based methods, proximity extension assays (e.g., the use of DNA oligonucleotides linked to antibodies against a target molecule that can be quantified with real-time polymerase chain reaction), slow-offrate-modified-aptamer reagent (SOMAmer) assays, nanoscale needles etc. Such technologies can include immunofluorescent assays, enzyme immunoassays, radioimmunoassays, chemiluminescent assays, sandwich-format assays, techniques using microfluidic or MEMS technologies, re-engineering technologies (e.g. instruments utilizing sensors for biomarkers used for telemedicine purposes), epitope-based technologies, other fluorescence technologies, microarrays, lab-on-a-chip, and rapid point-of-care screening technologies.

Exemplary proximity extension assays are available commercially from Olink (Uppsalla, Sweden), while exemplary slow-offrate-modified-aptamer reagent (SOMAmer) assays include SOMAscan™ assays available commercially from SOMAlogic (Boulder, CO). Exemplary assays using nanoscale needles that function as label free biosensors, functionalized with capture antibodies that change color once bound to its target (which is then quantified) is available commercially from companies such as NanoMosaic (Woburn, MA).

Other detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample can be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

“Protein biochip” refers to a biochip adapted for the capture of polypeptides. Many protein biochips are described in the art. These include, for example, protein biochips produced by Ciphergen Biosystems, Inc. (Fremont, Calif), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

In another embodiment, the biomarkers described herein are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. In one embodiment, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In another embodiment, the mass spectrometric technique for use in detecting the biomarkers described herein is “Surface Enhanced Laser Desorption and Ionization” or “SELDI.” This refers to a method of desorption/ionization gas phase ion spectrometry (e.g., mass spectrometry) in which the biomarkers are each captured on the surface of a SELDI mass spectrometry probe. There are several versions of SELDI known to those of skill in the art and contemplated for use herein, including “affinity capture mass spectrometry” or surface-enhanced affinity capture (SEAC), surface-enhanced neat desorption (SEND), surface-enhanced photolabile attachment and release (SEPAR).

In another embodiment, a mass-spectrometry method can be used to capture a biomarker on an appropriate chromatographic resin. For example, one could capture the biomarkers on a cation exchange resin, such as CM Ceramic HyperD F resin, wash the resin, elute the biomarkers and detect by MALDI. Alternatively, this method can be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI. These methods are known to those of skill in the art and are not described in detail herein.

In another embodiment, the biomarkers described herein can be detected and/or measured by immunoassay, where specific capture reagents, such as antibodies or binding fragments thereof, to bind each of the biomarkers in the panel. Antibodies can be produced by methods well known in the art, e.g., by separately immunizing animals with each of the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

Also specifically contemplated herein are traditional immunoassays for detecting or measuring the level of a biomarker in the panel including, but not limited to, sandwich immunoassays including ELISA or fluorescence-based immunoassays, as well as other enzyme immunoassays. In the SELDI-based immunoassay, an antibody or other binding reagent for the biomarker is attached to the surface of an MS probe, such as a pre-activated ProteinChip array. The biomarker can then be specifically captured on the biochip and detected by mass spectrometry.

Detection of a given biomarker may require the use of a label or detectable moiety. Such detectable moieties can be isotopic labels; magnetic, electrical or thermal labels; colored or luminescent dye; and enzymes, all of which enable detection of the biomarker(s). In various embodiments, a secondary detectable label is used. A secondary label is one that is indirectly detected including, but not limited to, one of a binding partner pair; chemically modifiable moieties; nuclease inhibitors; enzymes such as horseradish peroxidase, alkaline phosphatases, luciferases etc. In certain sandwich formats, an enzyme can serve as the secondary label, bound to the soluble capture ligand. In various embodiments, the system relies on detecting the precipitation of a reaction product or on a change on the properties of the label, for example the color for detection. A detection system for colorimetric methods can include a spectrophotometer, a colorimeter, or other device that measures absorbance or transmission of light on one or more wavelengths.

As described herein, assessment of results may be qualitative or quantitative depending upon the specific method of detection employed.

While the methods described herein are exemplified using analysis at the protein level, it is specifically contemplated herein that the step of determining expression of each member in a panel can be detected at the mRNA level can also be used with the methods described herein. In such an embodiment, the biological sample can comprise CSF, whole blood, serum, plasma, circulating RNA, exosomes and the like.

### Reference Levels

The results of an assay from a given biological sample can be compared to those of a control biological sample tested using substantially the same methods. Comparing the expression level or amount of each member in the panel of biomarkers in a biological sample from a subject suspected of having Alzheimer's disease with the expression level or amount of each member in the panel of biomarkers in a control biological sample can permit diagnosis or staging of Alzheimer's disease in the subject. Control biological samples can be a reference sample taken from the subject at an earlier time point (e.g., during a first episode of confusion or dementia-like symptoms) to permit monitoring of disease progress in the subject. For example, the control biological sample can be a sample taken from the subject one month, two months, three months, six months, or one year prior to the sample to be tested. In other embodiments, the control biological sample or reference sample is obtained from the subject during, or following the administration of a given Alzheimer's disease therapy. In other aspects, a reference sample can be a sample from a patient or a population of patients with no dementia. Alternatively, a reference sample can be a sample from a patient or population of patients with a known stage of Alzheimer's disease, for example, mild AD, moderate AD or advanced AD. In certain embodiments, a positive or negative control sample is a sample that is obtained or derived from a corresponding tissue or biological fluid or tumor as the sample to be analyzed in accordance with the methods as described herein. This sample will typically be from the same patient at the same or different time points.

Thus, in certain embodiments, a control biological sample is tested alongside the biological sample in the assays described herein. In some embodiments, the control biological sample is obtained from a normal or non-Alzheimer's subject or population of subjects and serves as a negative control or can be used for normalization to remove background noise from the assay. In other embodiments, the control sample can be obtained from the subject at an earlier time point, or a subject or population thereof known to have a certain degree of Alzheimer's disease (e.g., mild, moderate or severe disease). Such controls can serve as a positive control to ensure that the assay is working and/or permit “staging” of Alzheimer's disease. In other embodiments, one or more controls can comprise a known concentration (or range of concentrations) of each of the biomarkers in the panel in order to quantitatively detect the level of each biomarker in the subject being tested.

In certain embodiments the level of one or more of the biomarkers described herein can be compared to a reference value or the level the biomarker in a control or reference sample in order to assess the “risk” of a subject for developing Alzheimer's disease. Such reference levels can be from a subject or population of subjects that are cognitively normal, a subject or population of subjects previously diagnosed with AD (including subject(s) of a given stage of disease), or a previous sample from the same individual currently being tested. These reference levels can be used as a comparison for biomarker levels in samples (such as cerebrospinal fluid) obtained from an individual to assess risk of developing AD. An individual is determined to be at risk for or have AD if the level of one or more biomarkers are within reference levels of individuals with AD or outside reference levels of biomarkers for a normal population.

In another embodiment the reference levels for one or more biomarkers are established based on biomarker levels in a sample taken from an individual at an earlier point in time, for example, prior to onset of treatment with a therapeutic agent. The individual is determined to be responding to treatment for AD if the relative amounts of the biomarkers in the biological sample have altered favorably from the biomarker levels in a biological sample taken at an earlier first time point from the same individual; i.e. trend towards normal biomarker levels. Similarly, the disease state of the individual may be progressing if the biomarker levels in a biological fluid sample are changing relative to the levels in the individual taken at an earlier time point or in reference to the control levels.

The reference level or value can be derived from the level of protein expression of each member in the panel in a biological sample or population of biological samples. Alternatively, the reference level or value can be derived from the level of mRNA expression or each member in the panel in a biological sample or population of biological samples.

### Treatment of Alzheimer's Disease

Based on the diagnosis or staging of Alzheimer's disease determined using the methods described herein, a treatment for Alzheimer's disease is administered to a subject determined to have Alzheimer's disease. To date, the U.S. Food and Drug Administration (FDA) has approved two types of medications for the management of Alzheimer's disease: cholinesterase inhibitors, including Aricept® (donepezil), Exelon® (rivastigmine), Razadyne® (galantamine), and Cognex® (tacrine); and the NMDA-type glutamate receptor inhibitor memantine (marketed under a number of different brands). Although a cure for Alzheimer's disease has not been identified, these therapies serve to alleviate cognitive symptoms such as memory loss, confusion, and loss of critical thinking abilities in subjects diagnosed with age-related dementia (e.g., Alzheimer's disease).

In addition to these approved therapies, several studies indicate that pooled intravenous immunoglobulin (IVIG) is effective as a therapeutic in slowing the progression of symptoms in Alzheimer's patients (Dodel R C et al., J Neurol Neurosurg Psychiatry, October; 75(10):1472-4 (2004); Magga J. et al., J Neuroinflammation, December 7; 7:90 (1997); Relkin N R et al., Neurobiol Aging, 30(11):1728-36 (2008); Puli L. et al., J Neuroinflammation May 29; 9:105 (2012)). Thus, in one embodiment, a treatment for Alzheimer's disease to be used in conjunction with the methods described herein comprises administration of such intravenous immunoglobulin. Treatment with monoclonal antibodies against Aβ protein, such as bapineuzumab, ponezumab, and solanezumab are specifically contemplated herein.

Other exemplary treatments for AD include, but are not limited to, treatment with a cholinesterase inhibitor (e.g., donepezil, galantamine, or rivastigmine), aducanumab, or memantine.

In certain embodiments, the treatment for AD can comprise an anti-Tau therapy.

Also contemplated herein is identification and/or selection of a subject based on the methods described herein for participation in a clinical trial of an experimental treatment for AD. That is, experimental AD treatments are specifically contemplated herein for treatment of subjects diagnosed as having AD using the methods described herein.

### Pharmaceutical Compositions, Administration and Efficacy

Pharmaceutical or therapeutic compositions comprising a therapeutic agent for the treatment of Alzheimer's disease can contain a physiologically tolerable carrier, wherein the therapeutic agent is dissolved or dispersed therein as an active ingredient(s). In a preferred embodiment, the pharmaceutical composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological or pharmaceutical composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising a therapeutic agent for treatment of Alzheimer's disease can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of Alzheimer's disease or a symptom thereof will depend on the nature of the disorder or condition, and can be determined by standard clinical techniques.

Formulations for Particular Administration Routes: For buccal administration, the compositions can take the form of tablets or lozenges formulated in a conventional manner.

For administration by nasal inhalation, the therapeutic agent(s) are conveniently delivered in the form of an aerosol spray presentation from a pressurized pack or a nebulizer with the use of a suitable propellant, e.g., dichlorodifluoromethane, trichlorofiuoromethane, dichloro-tetrafluoroethane, or carbon dioxide. In the case of a pressurized aerosol, the dosage can be determined by providing a valve to deliver a metered amount. Capsules and cartridges of, for example, gelatin for use in a dispenser can be formulated containing a powder mix of the compound and a suitable powder base, such as lactose or starch.

A pharmaceutical composition as described herein can be formulated for parenteral administration, e.g., by bolus injection or continuous infusion. Formulations for injection can be presented in unit dosage form, e.g., in ampoules or in multidose containers with, optionally, an added preservative. The compositions may be suspensions, solutions, or emulsions in oily or aqueous vehicles, and can contain formulatory agents such as suspending, stabilizing, and/or dispersing agents.

Pharmaceutical compositions for parenteral administration include aqueous solutions of the active preparation in water-soluble form. Additionally, suspensions of the active ingredients can be prepared as appropriate oily or water-based injection suspensions.

Suitable lipophilic solvents or vehicles include fatty oils such as sesame oil, or synthetic fatty acid esters such as ethyl oleate, triglycerides, or liposomes. Aqueous injection suspensions can contain substances that increase the viscosity of the suspension, such as sodium carboxymethyl cellulose, sorbitol, or dextran.

Optionally, the suspension can also contain suitable stabilizers or agents that increase the solubility of the active ingredients, to allow for the preparation of highly concentrated solutions. Alternatively, the active ingredient can be in powder form for constitution with a suitable vehicle, e.g., a sterile, pyrogen-free, water-based solution, before use.

In some embodiments, a therapeutic agent can be delivered in an immediate release form. In other embodiments, the therapeutic agent can be delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the therapeutic agents, reduced dosage frequency, and increased compliance. In addition, controlled- or sustained-release compositions can favorably affect the time of onset of action or other characteristics, such as blood levels of the therapeutic agent, and can thus reduce the occurrence of adverse side effects.

Controlled- or sustained-release compositions can comprise an immediate release portion and an extended release portion. The immediate release portion immediately and acutely releases an amount of the therapeutic that promptly produces the desired therapeutic or prophylactic effect, while the extended release portion gradually and continually releases other amounts of the therapeutic agent to maintain a level of therapeutic or prophylactic effect over an extended period of time. Controlled- or sustained-release of an active ingredient can be stimulated by various conditions, including but not limited to, changes in pH, changes in temperature, concentration or availability of enzymes, concentration or availability of water, or other physiological conditions or compounds.

Controlled-release and sustained-release dosage forms can be used to provide controlled- or sustained-release of one or more active ingredients using, for example, hydroxypropylmethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known in the art, including those described herein, can be readily selected for use with the active ingredients of the invention in view of this disclosure. See also Goodson, “Dental Applications” (pp. 115-138) in Medical Applications of Controlled Release, Vol. 2, Applications and Evaluation, R. S. Langer and D. L. Wise eds., CRC Press (1984). Other controlled- or sustained-release systems that are discussed in the review by Langer, Science 249:1527-1533 (1990) can be selected for use according to the methods and compositions described herein. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of infection, e.g., skin, lungs, spinal column, brain, or gastrointestinal tract, thus requiring only a fraction of the systemic dose.

When in tablet or pill form, a pharmaceutical composition as described herein can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

The pharmaceutical composition as described herein can also be formulated in rectal compositions such as suppositories or retention enemas, using, for example, conventional suppository bases such as cocoa butter or other glycerides.

The appropriate dosage range for a given therapeutic agent depends upon the potency, and includes amounts large enough to produce the desired effect, e.g., reduction in at least one symptom of Alzheimer's disease or accompanying dementia in a subject. The dosage of the therapeutic agent should not be so large as to cause unacceptable or life-threatening adverse side effects. Generally, the dosage will vary with the type of inhibitor, and with the age, condition, and sex of the patient. The dosage can be determined by one of skill in the art and can also be adjusted by the individual physician in the event of any complication.

Typically, the dosage of a given therapeutic can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Currently available therapies, including experimental therapies, for Alzheimer's disease or a symptom thereof and their dosages, routes of administration and recommended usage are known in the art and/or have been described in such literature as the Physician's Desk Reference (60th ed., 2017). With respect to experimental therapies, an appropriate dosage can be estimated based on dose-response modeling in animal models or in silico modeling of drug effects.

Administration of the doses recited above or as employed by a skilled clinician can be repeated for a limited and defined period of time. In some embodiments, the doses are given once a day, or multiple times a day, for example but not limited to three times a day. Typically, the dosage regimen is informed by the half-life of the agent as well as the minimum therapeutic concentration of the agent in blood, serum or localized in a given biological tissue. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

A therapeutically effective amount is an amount of an agent that is sufficient to produce a statistically significant, measurable change of a given symptom of Alzheimer's disease or symptom thereof (see “Efficacy Measurement” below). Such effective amounts can be gauged in clinical trials as well as animal studies for a given agent. For example, reduction of a given symptom of Alzheimer's disease, dementia or impaired cognitive function, can be indicative of adequate therapeutic efficacy of an agent(s).

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one therapeutic agent can be conventionally administered in a unit dose. The term “unit dose” when used in reference to a therapeutic composition refers to physically discrete units suitable as unitary dosage for the subject, each unit containing a predetermined quantity of a therapeutic agent calculated to produce the desired therapeutic effect in association with the required physiologically acceptable diluent, i.e., carrier, or vehicle or in combination with a therapeutic agent useful for Alzheimer's disease.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

In some embodiments, a combination of therapeutic agents is used in the treatment of Alzheimer's disease in a subject diagnosed as described herein.

In some embodiments, a therapeutically effective agent is administered to a subject concurrently with a combination therapy. As used herein, the term “concurrently” is not limited to the administration of the two or more agents at exactly the same time, but rather, it is meant that they are administered to a subject in a sequence and within a time interval such that they can act together (e.g., synergistically to provide an increased benefit than if they were administered otherwise). For example, the combination of therapeutics may be administered at the same time or sequentially in any order at different points in time; however, if not administered at the same time, they should be administered sufficiently close in time so as to provide the desired therapeutic effect, preferably in a synergistic fashion. The agents can be administered separately, in any appropriate form and by any suitable route. When each of the therapeutic agents in a combination are not administered in the same pharmaceutical composition, it is understood that they can be administered in any order to a subject in need thereof. For example, the first therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second therapeutic agent, to a subject in need thereof (or vice versa). In other embodiments, the delivery of either therapeutic agent ends before the delivery of the other agent/treatment begins. In some embodiments of either case, the treatment is more effective because of combined administration. For example, the therapeutic agents used in combination are more effective than would be seen with either agent alone. In some embodiments, delivery is such that the reduction in a symptom, or other parameter related to the disorder is greater than what would be observed with either therapeutic agent alone. The effect of such a combination can be partially additive, wholly additive, or greater than additive. The agent and/or other therapeutic agents, procedures or modalities can be administered during periods of active disease, or during a period of persistence or less active disease.

When administered in combination, one or more of the therapeutic agents can be administered in an amount or dose that is higher, lower or the same as the amount or dosage of the a given agent used individually, e.g., as a monotherapy. In certain embodiments, the administered amount or dosage of a first therapeutic agent when administered in combination with a second therapeutic agent is lower (e.g., at least 20%, at least 30%, at least 40%, or at least 50%) than the amount or dosage of the first agent when used individually. In other embodiments, the amount or dosage of a first therapeutic agent, when administered in combination with a second therapeutic agent, results in a desired effect (e.g., improved cognitive functioning) is lower (e.g., at least 20%, at least 30%, at least 40%, or at least 50% lower) than the amount or dosage of the first (or second) agent required to achieve the same therapeutic effect when administered alone.

The efficacy of a given treatment for Alzheimer's disease can be determined by the skilled clinician. However, a treatment is considered “effective treatment,” as the term is used herein, if any one or all of the signs or symptoms of Alzheimer's disease or accompanying dementia is/are altered in a beneficial manner, or other clinically accepted symptoms or markers of disease are improved, or ameliorated, e.g., by at least 10% following treatment with a therapeutic agent for Alzheimer's disease. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the infection; or (2) relieving the disease, e.g., causing regression of symptoms; and (3) preventing or reducing the likelihood of the development of the disease, or preventing secondary diseases/disorders associated with the infection (e.g., secondary infections, sepsis etc.).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., pain, amount or presence of sputum, redness, localized swelling, fever, etc.

The invention may be as described in any one of the following numbered paragraphs:

1. A method for treating Alzheimer's disease in a subject, the method comprising: (i) receiving results of a protein assay relating to the expression of a panel of proteins comprising pyruvate kinase (PKM), fructose bisphosphate aldolase A (ALDOA), and lactate dehydrogenase B chain (LDHB) in a biological sample obtained from a subject, (ii) determining that the expression of at least one of the members of the panel of proteins is increased in the biological sample compared to the expression of the at least one member in a reference sample, and (iii) administering a treatment for Alzheimer's disease to the subject, thereby treating Alzheimer's disease in the subject.

2. A method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), and lactate dehydrogenase B chain (LDHB), (ii) determining that the expression of at least one of the members of the panel of proteins is increased in the biological sample as compared to the expression of the at least one member in a reference sample, and (iii) administering a treatment for Alzheimer's disease to the subject, wherein the treatment reduces or alleviates one or more symptoms of Alzheimer's disease in the subject.

3. The method of paragraph 1 or 2, wherein the biological sample is cerebrospinal fluid (CSF), whole blood, plasma, serum, or urine.

4. The method of any one of paragraphs 1-3, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

5. The method of any one of paragraphs 1-4, further comprising a step of obtaining the biological sample from the subject.

6. The method of any one of paragraphs 1-5, further comprising a step of selecting the subject based on the presence or expression level of apolipoprotein E isoform 4 (apoE4).

7. The method of any one of paragraphs 1-6, wherein the treatment for Alzheimer's disease comprises a cholinesterase inhibitor, intravenous immunoglobulin (IVIG), aducanumab, or memantine.

8. The method of paragraph 7, wherein the cholinesterase inhibitor comprises donepezil, galantamine, or rivastigmine.

9. The method of any one of paragraphs 1-8, further comprising repeating steps (i) and (ii) at a later time point after treatment has been initiated to monitor efficacy of the treatment.

10. A method for treating Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and 14-3-3 protein zeta/delta (YWHAZ), (ii) determining that expression of at least one of the members of the panel of proteins is increased in the biological sample as compared to the expression level of the at least one member in a reference sample, and (iii) administering a treatment for Alzheimer's disease to the subject, thereby treating Alzheimer's disease in the subject.

11. A method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and 14-3-3 protein zeta/delta (YWHAZ), (ii) determining that expression of at least one of the members of the panel of proteins in a biological sample is increased compared to the expression of the at least one of the members of the panel of proteins in a reference sample present, and (iii) administering a treatment for Alzheimer's disease to the subject, wherein the treatment reduces or alleviates one or more symptoms of Alzheimer's disease in the subject.

12. The method of paragraph 10 or 11, wherein the biological sample is cerebrospinal fluid (CSF), whole blood, plasma, serum, or urine.

13. The method of any one of paragraphs 10-12, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

14. The method of any one of paragraphs 10-13, further comprising a step of obtaining the biological sample from the subject.

15. The method of any one of paragraphs 10-14, further comprising a step of selecting the subject based on the presence or expression level of apolipoprotein E isoform 4 (apoE4).

16. The method of any one of paragraphs 10-15, wherein the treatment for Alzheimer's disease comprises a cholinesterase inhibitor, intravenous immunoglobulin (IVIG), aducanumab, or memantine.

17. The method of paragraph 16, wherein the cholinesterase inhibitor comprises donepezil, galantamine, or rivastigmine.

18. The method of any one of paragraphs 10-17, further comprising repeating steps (i) and (ii) at a later time point after treatment has been initiated to monitor efficacy of the treatment.

19. A method for treating Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and apolipoprotein E isoform 4 (apoE4), (ii) determining that the expression of at least one of the members of the panel of proteins is increased in the biological sample as compared to the expression of the at least one member in a reference sample, and (iii) administering a treatment for Alzheimer's disease to the subject, thereby treating Alzheimer's disease in the subject.

20. A method for reducing or alleviating one or more symptoms of Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and apolipoprotein E isoform 4 (apoE4), (ii) determining that the expression of at least one of the members of the panel of proteins is increased in the biological sample as compared to the expression of the at least one member in a reference sample, and (iii) administering a treatment for Alzheimer's disease to the subject, thereby treating Alzheimer's disease in the subject.

21. The method of paragraph 19 or 20, wherein the biological sample is cerebrospinal fluid (CSF), whole blood, plasma, serum, or urine.

22. The method of any one of paragraphs 19-21, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

23. The method of any one of paragraphs 19-22, further comprising a step of obtaining the biological sample from the subject.

24. The method of any one of paragraphs 19-23, wherein the treatment for Alzheimer's disease comprises a cholinesterase inhibitor, intravenous immunoglobulin (IVIG), aducanumab, or memantine.

25. The method of paragraph 24, wherein the cholinesterase inhibitor comprises donepezil, galantamine, or rivastigmine.

26. The method of any one of paragraphs 19-25, further comprising repeating steps (i) and (ii) at a later time point after treatment has been initiated to monitor efficacy of the treatment.

27. A method for treating Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, and (iii) administering a treatment for Alzheimer's disease to the subject, thereby treating Alzheimer's disease in the subject.

28. A method for reducing or alleviating a symptom of Alzheimer's disease in a subject, the method comprising: (i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, and (iii) administering a treatment for Alzheimer's disease to the subject, thereby treating Alzheimer's disease in the subject.

29. The method of paragraph 27 or 28, wherein the biological sample is cerebrospinal fluid (CSF), whole blood, plasma, serum, or urine.

30. The method of any one of paragraphs 27-29, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

31. The method of any one of paragraphs 27-30, further comprising a step of obtaining the biological sample from the subject.

32. The method of any one of paragraphs 27-31, wherein the treatment for Alzheimer's disease comprises a cholinesterase inhibitor, intravenous immunoglobulin (IVIG), aducanumab, or memantine.

33. The method of paragraph 32, wherein the cholinesterase inhibitor comprises donepezil, galantamine, or rivastigmine.

34. The method of any one of paragraphs 27-33, further comprising repeating steps (i) and (ii) at a later time point after treatment has been initiated to monitor efficacy of the treatment.

35. A method for diagnosing Alzheimer's disease in a subject, the method comprising: (i) receiving results of a protein assay relating to the expression of each member of a panel of proteins in a biological sample obtained from a subject, wherein the panel of proteins comprises at least two of the members of, or consists essentially of, pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, thereby diagnosing the subject as having Alzheimer's Disease.

36. A method for selecting a subject to be treated for Alzheimer's disease, the method comprising: (i) receiving results of a protein assay relating to the expression of each member of a panel of proteins in a biological sample obtained from a subject, wherein the panel of proteins comprises at least two of the members of, or consists essentially of, pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4), (ii) determining that the expression of at least one of the members of the panel of proteins is increased in the biological sample as compared to the expression of the at least one member in a reference sample, thereby selecting the subject to be treated for Alzheimer's Disease.

37. The method of paragraph 36, wherein the subject is selected for participation in a clinical trial for an Alzheimer's disease agent.

38. The method of paragraph 36 or 37, wherein the Alzheimer's disease agent is an early-stage intervention for Alzheimer's disease.

39. The method of any one of paragraphs 2, 11, 20 or 28, wherein the one or more symptoms of Alzheimer's disease comprise: impaired cognitive function, worsening cognitive function, paranoia, episodes of forgetting, confusion, disorientation, agitation, irritability, depression, hallucinations, difficulty concentrating, impaired mathematical reasoning, impaired ability to form new memories, anger or combinations thereof.

### EXAMPLES

### Example 1

Summary: Provided herein are methods for diagnosing and treating Alzheimer's disease based on the meta-analysis of published proteomics datasets from multiple publications to mine for cerebrospinal fluid (CSF)-based diagnostic biomarkers for Alzheimer's disease (AD). Biomarkers identified using these methods were validated in an independent cohort analyzed in house and two additional published large datasets. Specifically, published data from 6 independent and a 7^{th} in-house acquired CSF proteomic datasets were mined resulting in a meta-cohort with 73 AD cases and 77 controls. In-depth data analysis revealed 35 CSF biomarker candidates, many of which are associated with brain glucose homeostasis, which is known to be dysregulated in AD. Next, the list of identified biomarker candidates was pruned and validated in a 2-pronged approach using i) an independent cohort analyzed in house, and ii) two recently published independent large scale datasets comprising in total more than 500 samples. The resulting biomarker panel consisting of three glycolytic enzymes was found to discriminate AD from controls. To the inventor's knowledge, this is the first study that describes the mining and systematic re-analysis of previously published liquid chromatography mass spectrometry-based proteomics data to identify and validate biomarkers in general and CSF biomarkers for AD in particular. While the presented study focused on AD, the presented workflow can be applied to any disease for which published datasets are available.

### INTRODUCTION

Alzheimer's disease (AD) is the most prevalent form of dementia known for its gradual deterioration of cognitive functions, such as memory, thinking, and reasoning, as well as psychological changes including depression and behavioral changes (1). Differentiation of AD from other types of dementia has been impeded by the variability of clinical symptoms both within and between dementias (2). The lack of inexpensive and large-scale application of in vivo diagnostics has focused research on the detection of biomarkers in body fluids, especially cerebrospinal fluid (CSF) due to its proximity to and interactions with the brain (3, 4). Numerous proteomics studies have attempted to discover novel CSF-based AD biomarkers. Although these studies have yielded several biomarker candidates, only a few replication studies exist and little overlap in these sets of AD biomarker candidates has been observed (5-13). Possible explanations for this observed lack in overlap include small cohort sizes, inconsistency in diagnostic criteria for dementia, variability in sample handling and differences in data acquisition and analysis. To fill this gap, the inventors performed meta-analysis of raw liquid chromatography mass spectrometry (LC/MS) data from several independent CSF proteomics studies aimed at identifying AD biomarkers. It was hypothesized that this approach would not only overcome some of these limitations and increase the statistical power and precision but also broaden the applicability of any discovery to diverse populations by inclusion of numerous independent cohorts. To test this hypothesis, a meta-analysis of 6 previously published studies and one in-house dataset was carried out on quantitative CSF proteomics in the context of AD to identify biomarker candidates which were validated using a 2-pronged approach: firstly, by analyzing CSF specimens from an 8^{th} independent cohort, and secondly, by querying two recently published large-scale CSF proteomics studies aimed at identifying biomarkers for AD (14, 15).

### RESULTS AND DISCUSSION

Data Pre-processing: The inventors searched PubMed and retrieved 394 potentially relevant proteomic AD-focused biomarker discovery studies (FIG. 1A). Six of these met the stringent inclusion and exclusion criteria (see Materials and Methods) including the availability of raw high resolution/high accuracy LC/MS data (Table 3).

TABLE 3
Information about the six studies with associated AD CSF MS data selected for re-analysis. Information
about the origin of the samples, the number of samples for AD and control, the type of MS instruments
used, quantification methods and information about enrichment of the samples.

| | Country of | | # of | | | | | |
|---|---|---|---|---|---|---|---|---|
| | Sample | # AD | control | Disease | Control | Type of | Peptide | |
| Reference | Origin | samples | samples | definition | definition | MS | labelling | Enrichment |
| Dayon et al. | Switzerland | 30 | 30 | CSF P- | Healthy | Orbitrap | TMT 6 | MARS14 |
| [16] | | | | tau181/Aβ1- | controls, | Elite | plex | |
| | | | | 42 ratio >0.0779 | CDR = 0 | | | |
| Sathe et al. | USA | 5 | 5 | In house | Healthy | Orbitrap | TMT 10 | MARS14 |
| [17] | | | | classification. | controls, | Fusion | plex | |
| | | | | Similar to | CDR = 0 | Lumos | | |
| | | | | NINCDS- | | | | |
| | | | | ADRDA | | | | |
| Khoonsari et | Sweden | 10 | 10 | Brain | Control had | 7T LTQ- | None | MARS-Hu7 |
| al. [18] | | | | imaging, | normal | FT | | |
| | | | | laboratory | cognition | | | |
| | | | | testing, | according to | | | |
| | | | | neurological | MMSE | | | |
| | | | | and | performance | | | |
| | | | | cognitive | | | | |
| | | | | examinations | | | | |
| Lleó et al. | Spain | 1* | 3* | NINCDS- | Assessed by | Orbitrap | None | Albumin & |
| [19] | | | | ADRDA | neurologist | Velos Pro | | IgG |
| Barucker et | Germany | 19 | 20 | MMSE/ | Age-matched | Orbitrap | None | No |
| al. [20] | | | | MRI | | Velos | | information |
| | | | | classification | | | | |
| Wang et al. | USA | 4 | 4 | NINCDS- | Controls had | Q | None | Glycoproteomics |
| [21] | | | | ADRDA | normal | Exactive | | |
| | | | | | cognition | | | |
| | | | | | according to | | | |
| | | | | | MMSE | | | |
| | | | | | performance | | | |

*The samples that were used by Lleó et al. (2019) are pooled samples comprising 10 CSF samples per pool. National Institute of Neurological and Communicative Disorders and Stroke and the Alzheimer's Disease and Related Disorders Association (NINCDS-ADRDA), clinical dementia rating (CDR), Mini-mental state exam (MMSE).

Supplementing these six datasets with one additional in-house dataset resulted in a total of 150 samples (73 AD, 77 Control) derived from seven independent cohorts from five countries on both sides of the Atlantic (FIG. 1A) (16-21). All retrieved raw LC/MS data were re-analyzed using MaxQuant followed by data normalization using either the median of summed intensities of all samples (if no reference samples were available) or the average of the median intensities of reference sample (FIGS. 1B & 1C). Next, an in-house developed outlier detection method was applied to each sample set individually; this method correlates all protein intensities from each sample with the sample set-specific median intensities. Samples with correlation values 3 standard deviations from the mean were classified as outlier and removed from further analysis (FIG. 1D). This step led to the removal of 13 samples from the seven datasets resulting in 65 AD cases and 72 controls. Manual inspection of the outlier detection method in principal component analysis (PCA) provided evidence that the objective outlier detection method was effective for the removal of outliers. Further, the method is easily tuneable and can be adjusted to be more or less stringent. For example, if one can test many potential biomarkers in future studies, less stringent criteria can be used. On the other hand, if antibodies need to be developed for validation then higher stringency might be more appropriate.

Due to the differences in instrumentation and methodology used for each of the datasets, a direct comparison of the reported intensity values was not possible as evidenced in the PCA plot shown in FIG. 1E where the use of raw intensity values completely separates the different datasets. To overcome this problem of incompatible raw intensity values, each dataset was Z-score normalized based on the control samples within each sample set. PCAs of the data before and after the z-score normalization are shown in FIGS. 1E and 1F, clearly showing the need and suitability of the chosen z-score normalization procedure.

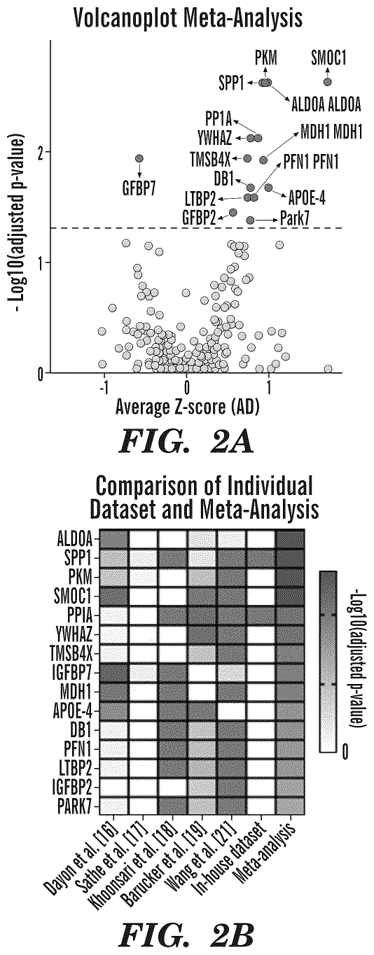

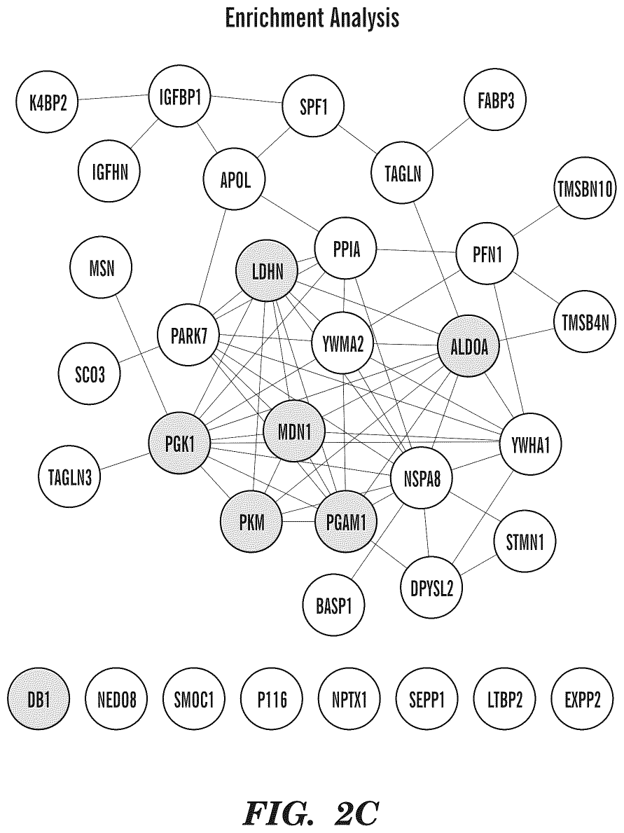

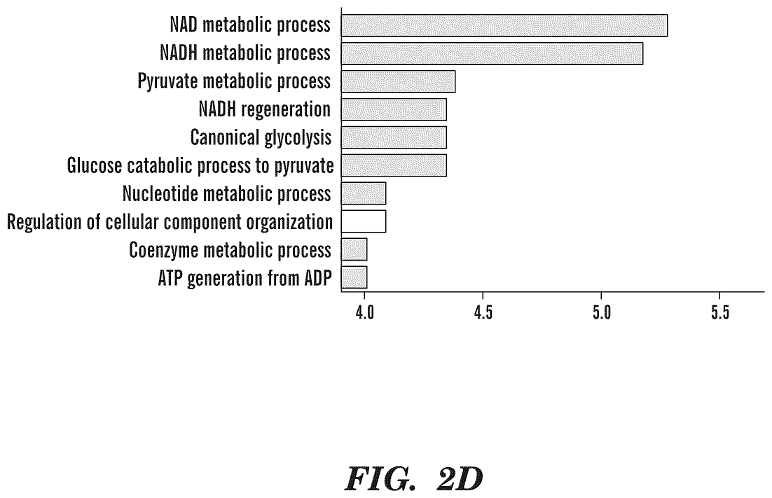

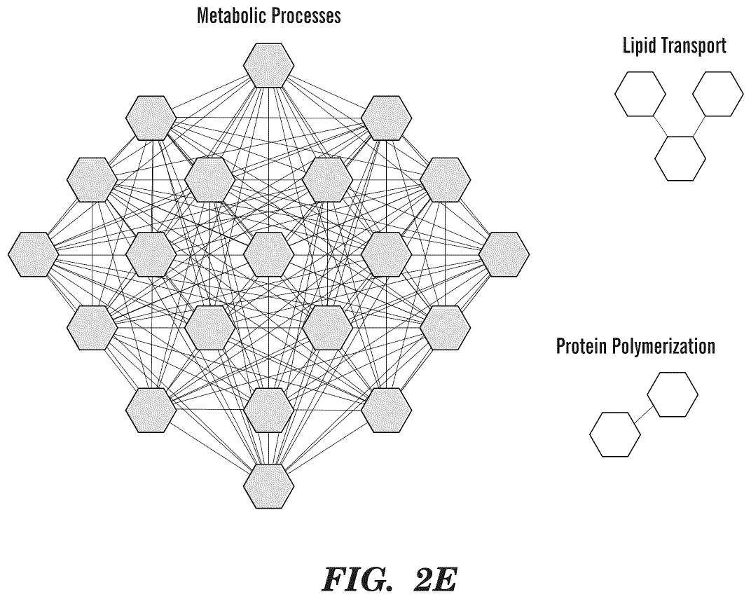

Discovery of Biomarker Candidates: The curated combined dataset is comprised of 137 samples with a total of 2899 identified proteins. First, a Benjamini-Hochberg-corrected Fisher Exact test was performed to identify proteins specifically detectable in only either AD cases or controls. This analysis did not result in any significant proteins when using an adjusted p-value of <0.05 as cut-off value. Next, all proteins not observed in at least 50% of samples were removed resulting in a list of 507 proteins, which were analyzed with the non-parametric Mann-Whitney U-test followed by Benjamini-Hochberg correction for multiple testing. This analysis step resulted in 15 proteins with statistically significant abundance differences between AD and controls (FIG. 2A). Interestingly, some of the proteins that were found to be significant in the meta-analysis were not found to be significant in the individual datasets (FIG. 2B), supporting the hypothesis that the meta-analysis of multiple independent datasets can reveal novel biomarker candidates.

Next, it was hypothesized that given the heterogeneity in study samples some proteins might be associated with disease status only in some of the datasets. To evaluate the effect of the individual datasets on the meta-analysis, each dataset was removed from the combined dataset prior to a repeat of the above described statistical analysis. This procedure was repeated 7 times with each dataset being absent once. This analysis led to the identification of an additional 20 proteins that show significant differential abundance between AD and control subjects (Table 4). Combining these proteins with the 15 from the analysis of the complete dataset resulted in a total of 35 candidates for CSF-based AD biomarkers. Some of the biomarker candidates have been linked to AD before supporting the data mining strategy: in a study of AD mutation carriers, PKM and ALDOA were found to be significantly enriched in the carrier group (12). Another targeted proteomics study found SPP1, MDH1, APOE, ENPP2 and IGFBP2 to be significant in AD CSF compared to control subjects (22). In contrast, some proteins that are often considered promising candidates such as YKL-40, neurofilament light or neurosecretory protein VGF were not found to be significant in this meta-analysis (23-25). This inconsistency may suggest that biomarker candidate identifications should be based on several independent cohorts and/or studies. Furthermore, it highlights the power of analyzing larger numbers of patients from a larger number of independent cohorts that are increasingly available in public data repositories.

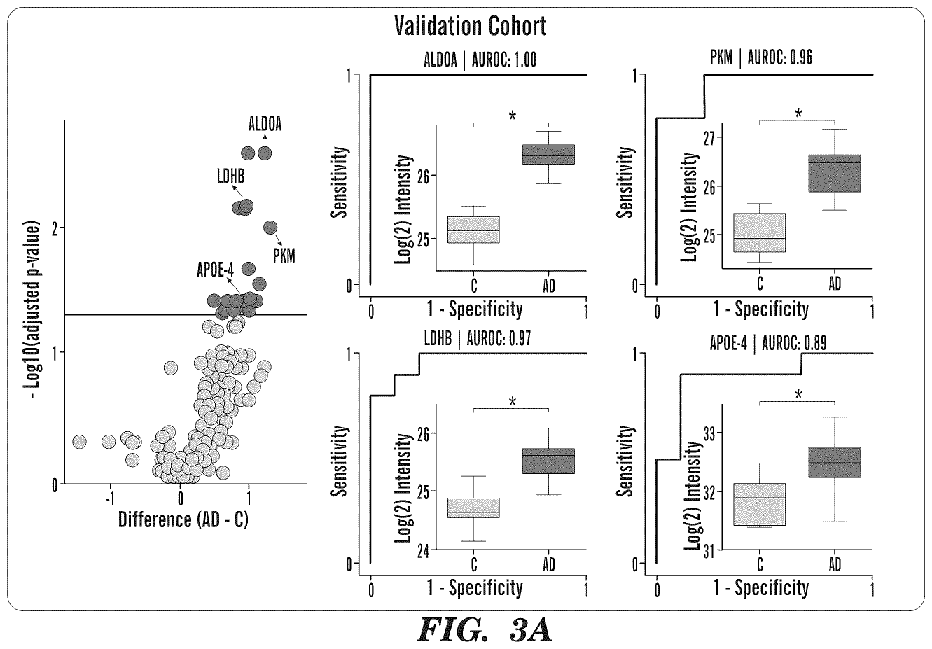

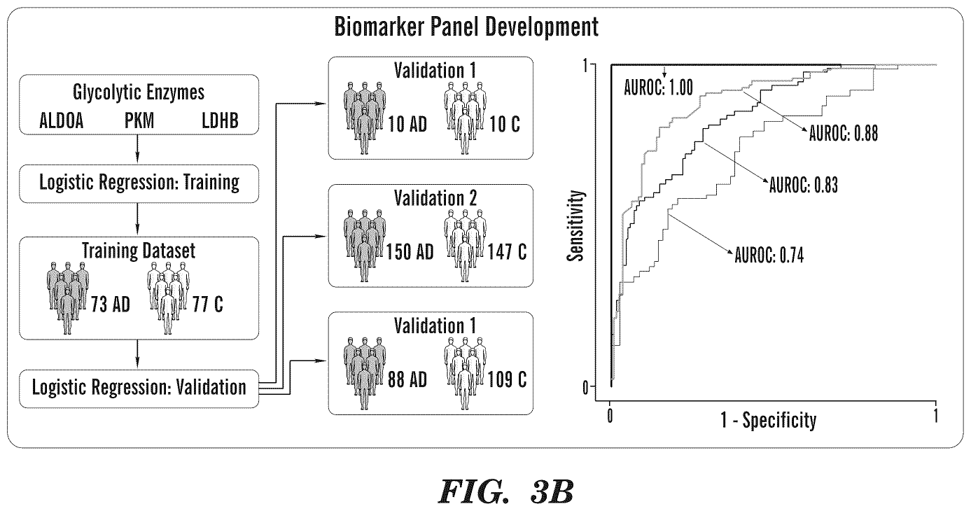

Bioinformatic Analysis of Biomarker Candidates: To determine known physical and functional interactions of the potential biomarkers identified, the STRING tool was used for the 35 biomarker candidates (26). Interestingly, the protein-protein network (FIG. 3C) shows a well-interconnected protein network for more than three-quarters of the identified biomarker candidates. In addition, 166 significant GO/biological process annotations (FDR<0.05) were identified, of which the top 10 are shown in FIG. 3D. It is noteworthy that 9 out of the top 10 GO annotations were associated with redox metabolic and glycolysis processes (highlighted in orange). Due to the large number of significant GO annotations by STRING, ClueGO™ was used to cluster these significantly enriched GO annotations (FIG. 3E), resulting in a highly interconnected cluster of GO annotations related to metabolic processes, underscoring the importance of perturbed metabolism and redox processes in AD. This finding underscores the paradigm that biomarkers should be a reflection of the disease-relevant pathophysiological processes going awry upon onset of a disease. This finding further supports the validity of the identified biomarker candidates given that dysregulation of redox processes and mitochondrial function in the AD diseased brain tissue is well-documented (14, 15, 27-35).

Biomarker Validation: To validate the 35 biomarker candidates, a 2-pronged approach was used. Firstly, an independent cohort of 20 (10 AD and 10 non-AD control) samples from Gothenburg, Sweden was analyzed. Both, the AD and the control group comprised five males and five females each. Analysis by unbiased discovery LC-MS without any depletion and/or fractionation resulted in the identification of 433 proteins in CSF. The same workflow that was used for the meta-analysis was followed, i.e., data normalization, test for outliers, Fisher Exact test (which did not identify any AD or control-specific proteins), and filtering based on completeness.

The statistical analysis of the validation cohort using Benjamini-Hochberg-corrected Mann-Whitney U-test resulted in a set of 24 proteins that were significantly different between AD and control CSF (adjusted p-value<0.05). Of these 24 proteins, four overlapped with the biomarker candidates identified in the meta-analysis, i.e., providing an initial validation of them as CSF biomarkers for AD (FIG. 3A): ALDOA, PKM, LDHB and APOE-4. All four biomarker candidates individually showed excellent performance separating AD from controls as assessed by receiver-operating characteristic (ROC) analysis with areas under the ROC curves (AUROC) ranging from 0.89 to 1.0, confirming these proteins as CSF biomarker candidates for AD (FIG. 3A). It was noteworthy that apart from APOE-4, a well-established AD-associated protein, the other three confirmed biomarker candidates (ALDOA, PKM and LDHB) are glycolytic enzymes which are linked to redox metabolic processes, underscoring the importance of this pathway in the pathogenesis of AD and its potential for biomarkers. The performance of these four validated biomarker candidates were tested in the original composite discovery cohort comprised of 7 different sub cohorts. The individual proteins resulted in AUROCs between 0.64 and 0.71, while a logistic regression using the four confirmed biomarker candidates (ALDOA, PKM, LDHB and APOE4) resulted in a AUROC of 0.79 in the meta-analysis of this highly heterogeneous group of datasets.

A second, independent validation of the panel of biomarkers candidates was carried out, leveraging two recently published large CSF proteomics studies aimed at identifying AD biomarkers (14, 15). Even though both were significantly better powered studies, only 4 out of the top 10 proteins (based on AUROC) were consistent across those two studies (14-3-3 protein zeta/delta (YWHAZ), ALDOA, PKM, L-lactate dehydrogenase A chain (LDHA)), highlighting the problem of cohort and analysis heterogeneities discussed above. Even so, 2 out of these 4 proteins were consistent with the biomarker candidate panel (ALDOA, PKM). LDHB was amongst the top 6 and top 18 in the studies by Johnson et al. (2020) and by Bader et al. (2020), respectively, while APOE-4 did not show up in either top 10 list. Given this absence of APOE-4, a biomarker panel was developed that consisted of the three glycolytic enzymes ALDOA, PKM and LDHB. The panel was modelled using training logistic regression analysis followed by applying them to three independent validation cohorts (in house, Johnson, Bader), resulting in AUROCs of 1.00, 0.88 and 0.83, respectively (FIG. 3B), which is consistent with the AUROCs of YWHAZ (0.90 and 0.84, respectively). YHWAZ was the best performing protein across the Johnson and Bader studies, but did not meet the stringent validation criteria in this study, i.e., being confirmed in the first validation cohort. These results strongly support the original hypothesis that the in silico combination of multiple small datasets into one larger one enables a significantly improved biomarker discovery effort.

An extensive body of research identifying CSF proteins associated with AD has failed to yield consensus biomarkers or panels there-of, most likely due to small sample size and lack of independent cohorts for validation. To overcome these limitations, the inventors combined the data from several small independent studies into one larger study comprising numerous independent cohorts. To overcome problems associated with the comparisons of independent datasets, a robust strategy was developed to combine publicly available raw LC/MS datasets and quantitatively re-analyze them to identify statistically more significant biomarker candidates. Using the meta-analysis strategy, a multi-cohort CSF proteomic dataset including 77 AD cases and 73 controls from 7 independent cohorts enrolled in 5 different countries resulted in 35 CSF biomarker candidates for AD, with the majority pointing to the well-described dysregulation of the redox metabolic pathways in AD, underscoring the notion that CSF biomarkers can reflect pathophysiology observed in the brain.

Pursuing a 2-pronged validation approach, first, the CSF proteome of an independent 8^{th} AD-focused cohort was mapped, which confirmed 4 of the 35 biomarker candidates. Second, a biomarker panel was developed using ALDOA, PKM and LDHB, which was applied to two recent large-scale CSF proteomics studies aimed at AD biomarker which proved to be effective in differentiating 321 AD CSF samples from 343 control samples. This panel, which is composed of three glycolytic enzymes, is indicative of dysregulated metabolism and could be a key diagnostic tool for differentiating AD from various other dementias or neurodegenerative diseases in living patients. A well-designed validation cohort that includes healthy controls as well as symptomatic controls, i.e. patients with non-AD dementias will be the next step for fully establishing the clinical usefulness of the candidate biomarker panel for AD. To the inventors' knowledge, this is the first biomarker discovery study that describes the collection and quantitative re-analysis of raw numerous independent LC-MS-based proteomic datasets. The workflow described herein can provide a general guideline for the quantitative re-analysis of proteomic data that are publicly available, particularly to enhance biomarker discovery studies and increase the statistical power by combining several independent cohorts and by increasing the number of samples.

### Biomarker Discovery

Literature Review: To retrieve AD-related CSF proteomics datasets, PubMed was searched with multiple combinations of the following keywords: ‘Alzheimer's disease’, ‘biomarker discovery’, ‘cerebrospinal fluid’, ‘proteomics’, ‘dementia’, ‘mass spectrometry’, ‘discovery proteomics’ and ‘neurodegeneration’. The resulting PubMed search results were downloaded and reviewed to determine if a paper described a study of CSF proteomes from AD patients using data-dependent acquisition (DDA) mode.

Exclusion criteria were: published before 2010; reviews (systematic or literature); written in other languages than English; no CSF proteomics; non-human CSF; no AD-related samples; only post mortem CSF; CSF not collected by a lumbar puncture; no information about the origin of samples; no description of sample preparation and/or MS techniques; use of non-DDA methods (e.g. SRM, MRM, PRM, Western Blot, 2D gel electrophoresis); no information about AD diagnosis criteria or CSF collection; peptidomics for biomarker discovery, isobaric labelling at the protein level or use of low resolution/low accuracy MS instrumentation.

Inclusion criteria were: published between Jan. 1, 2010 and Jan. 31, 2019; proteomic analyses of CSF from AD patients and in controls; CSF collection ante mortem by lumbar puncture; proteomic profiling using LC-MS/MS operated in DDA mode; use of high resolution/high accuracy instrumentation; well defined and described AD diagnosis and a clear definition of controls.

The selected papers were searched to determine if the paper describes the availability of the raw MS data in repositories such as PRIDE or MassIVE (massive.ucsd.edu)(36). If data were not available on repositories, authors were contacted directly requesting their raw LC-MS data. In some cases, LC-MS data were available, but the relevant meta-data was not. Some of these complications could be resolved by directly contacting the corresponding authors. If several methods were described in a single publication only the dataset with the largest number of identified proteins was used for further analysis.

One (unpublished) additional in-house dataset was used in this study. Quantitative proteomic mapping of these samples was performed using tandem mass tags (TMT) and analyzed on a Q Exactive Hybrid Quadrupole-Orbitrap mass spectrometer (ThermoFisher, Waltham, MA, USA). Similar to the publications selected in the literature review the CSF of the in-house dataset was collected ante mortem.

MaxQuant: All downloaded raw LC/MS data were analyzed in MaxQuant 1.6.1. using the human UniprotKB/Swiss-prot protein sequence database which was downloaded on Jan. 17, 2019 (37). The Uniprot database included all isoforms as well as the three APOE isoforms APOE2, APOE3 and APOE4, resulting in 42432 protein entries. In MaxQuant, standard settings were used including a first search with a 20 ppm mass tolerance and a main search of 4.5 ppm mass tolerance. A maximum of three tryptic missed cleavages was allowed. The following modification settings were used: carbamidomethylated cysteine residues (fixed), acetylation of the N-terminal of proteins (variable) and oxidation of methionine (variable). For studies that used TMT labelling, TMT 6-plex or TMT 10-plex (+229.163 Da) modification at the N terminus of the peptide as well as at lysine were set as fixed modifications. For non-TMT studies LFQ was turned on with the fast LFQ setting turned off A 1% False Discovery Rate (FDR) was used for the identified proteins. The match between runs feature was turned on in MaxQuant with standard parameters.

Data normalization: Protein identification and quantification outputs were exported from the MaxQuant-generated ‘proteingroups’ file which was loaded into Spyder (Python 3.6). Because some of the datasets included a reference node, two normalization methods were scripted in Python. If there was no reference node in the data, the summed intensity for each sample was calculated followed by the calculation of the median of all summed intensities for each study individually. Next, for each sample in the dataset the normalization factor (NF) was calculated by dividing the median of all summed intensities by the summed intensity of a given sample. Subsequently, this factor was used to normalize the protein intensities of the corresponding sample. If a dataset did include one or more reference sample the median intensity of the reference sample was calculated and used to determine the NF. In the case of multiple reference nodes, the average of all median intensities was calculated and used to determine the NF. The NF was calculated by dividing the median intensity of the reference node (or average if multiple reference nodes) by the median intensity of a given sample. The NF was subsequently used to normalize the protein intensities of the corresponding sample.

Outlier removal: In shotgun MS proteomic studies there is no standardized manner to select and remove samples that should be considered outliers. Usually, principal component analysis (PCA) plots of the samples are created, and samples are considered outlier based on their location relative to the other samples. This subjective approach can then lead to the removal of the outlier from datasets but lack reproducibility. For the re-analysis of datasets, this approach was avoided and a standardized approach was developed for outlier identification instead. The normalized datasets were loaded into Spyder where a theoretical sample was created based on the median intensities calculated for each protein identified in the respective dataset. Next, the Pearson correlation between each sample and this theoretical median sample was calculated in each dataset. Next, the standard deviations for the correlation coefficients were calculated and any sample with a correlation coefficient more than three standard deviations away from one was considered an outlier and removed from the dataset. A similar approach using three standard deviations as a criterion for being an outlier can be found in the paper by Spellman et al. (38).

Z-score data transformation: The collected datasets from different labs used different methodologies and hardware set-ups for their proteomic profiling of the CSF samples. Therefore, the intensities of the different datasets were not directly comparable; instead, the Z-scores were calculated to allow for comparisons. For the Z-score transformation, all data were first loaded into Spyder where all zero intensities produced by MaxQuant were treated as missing values, i.e. these values were removed from the intensity matrix. Subsequently, all remaining intensity values were log 2- and then Z-score transformed: For a given protein in each dataset, the mean and standard deviation were calculated based on the intensities of the control samples in a dataset. These values were then used to calculate the Z-scores of the corresponding protein for all samples (Z-score=(intensity−mean)/standard deviation). This process was then repeated for all protein across all datasets. Finally, all datasets were combined into one large dataset. The effectiveness of this procedure was confirmed by comparing the PCA plots before and after Z-score transformation.

To create PCA plots, imputation of missing values was required which was carried out in the Perseus environment (39). First, proteins with more than 30% missing values were removed from the datasets. Next, missing values were imputed by random numbers that were drawn from a normal distribution (0.3 width and 1.8 downshift for untransformed data, 0.3 width and 0 downshift for Z-score transformed data). The imputed datasets were exported which were solely used for the creation of the PCA plots and not for any other downstream analysis. The exported datasets were loaded into Spyder where PCA plots were created with the Seaborn-package (40).

Statistical analysis: Due to high variability and/or incompleteness of meta data such as age, sex, or Braak stages, it was only possible to test for differences in proteins between AD and controls without control for potential confounders. All statistical analysis was executed in R-studio (41). First, a Fisher exact test was used to identify proteins that might show statistical significance due to the percent presence/absence in the AD vs. control group. Next, proteins with more than 50% missing values were removed from the dataset. For statistical analysis, the non-parametric Mann-Whitney U test was used. The resulting p-values were corrected for multiple comparison using the Benjamini-Hochberg procedure (42). Significant proteins (adjusted p-value<0.05) were considered biomarker candidates and were visualized with the R package ‘EnhancedVolcanoplot’ (43). To test for the possibility that one dataset has a strong influence on the results in such a manner that an otherwise significant protein suddenly becomes insignificant (or vice versa), the combined dataset was re-analyzed with the Mann-Whitney U test and Benjamini-Hochberg correction after removing one dataset at a time. The additional proteins, that were found to be significant after removing one dataset at a time, were considered biomarker candidates, and used for down-stream analysis. Last, since it was hypothesized that the meta-analysis would results in more biomarker candidates compared to analyzing each dataset separately, we analyzed each of the datasets used in this meta-analysis on its own with a Student's T-test and Benjamini-Hochberg correction. The results were used to create a heatmap (Graphpad Prism 8.3.0., GraphPad Software, La Jolla, CA, USA) where the p-values of the found biomarker candidates of the meta-analysis were compared with the p-values of the biomarker candidates when datasets were analyzed on their own.

Functional enrichment analysis: The identified biomarker candidates were analyzed to determine their functional enrichment. First, STRING analysis was used to create a protein-protein interactions network, which was further analyzed to determine the biological process GO annotation that was associated with the protein-protein network (26, 44, 45). The biological process GO annotation data was isolated from STRING where the top 10 significant enriched annotations (FDR<0.05) were visualized with the Seaborn package in Spyder. Due to the large number of significantly found GO annotations we further analyzed the biomarker candidates with the Cytoscape plug-in ClueGO (46). ClueGO can compare and integrate clusters/groups of GO annotations based on kappa statistics to connect GO terms to one another.

### Validation of Biomarker Candidate

Sample handling: For the subsequent validation, 20 CSF samples provided by the Zetterberg lab (University of Gothenburg, Mölndal, Sweden) were used. The samples were from patients who sought medical advice because of cognitive impairment. Patients were designated as normal (n=10) or AD (n=10) according to CSF biomarker levels, measured using INNOTEST assays (Fujirebio, Ghent, Belgium), using cut-offs that are >90% specific for AD: total tau (T-tau)>350 ng/L and Aβ42<530 ng/L (47). None of the biochemically normal subjects fulfilled these criteria. To collect the CSF, lumbar punctures were performed in the morning. CSF was stored in polypropylene tubes and centrifuged to pellet any cell debris. After centrifugation, all CSF samples were frozen and stored at −80° C. without thawing until the experiment. The Regional Ethics Committee at the University of Gothenburg approved the study.

Sample Processing, Digestion and Clean-up: CSF samples were prepared for proteomic analysis using an in-house-developed MStern Blotting protocol which was adapted for CSF samples (48-50). Briefly, 100 μL of CSF samples were processed using a PVDF 96-well membrane plate (Merck-Millipore, MA, USA). Initially, the 100 μL of CSF was mixed with 100 μL urea buffer (8 M in 50 mM ammonium bicarbonate (ABC). To further reduce the disulphide bonds on the proteins 30 μL Dithiothreitol (DTT) (0.05 M in water) was added and incubated in a thermomixer (300 rpm) for 20 minutes at room temperature. To prevent the re-formation of disulphide bonds, 30 μL Iodoacetamide (IAA) (0.25 M in water) was added and incubated in a thermomixer (300 rpm) for 20 minutes at room temperature in the dark.

Reduced and alkylated CSF protein suspension was transferred to a 96 well polyvinylidene fluoride (PVDF) membrane (MSIPS4510, Millipore, MA, USA), which had been activated with 150 μL 70% ethanol and subsequently primed with 200 μL of urea buffer. To facilitate the transfer of the solution through the PVDF membrane a vacuum manifold was used. CSF proteins are captured on the PVDF membrane and were washed with 200 μL 50 mM ABC before applying 100 μL digestion buffer (0.4 μg Trypsin (V5111, Promega, WI, USA) in 50 mM ABC) to the 96-wells plate. The 96-wells plate was wrapped in parafilm and put in a 37° C. dark humidified incubator for two hours to facilitate digestion of the proteins. After incubation, the remaining digestion buffer was evacuated from the 96-wells PVDF membrane plate using a vacuum manifold. Proteins, now peptides, were eluted twice with 150 μL of 40% acetonitrile (ACN), 0.1% formic acid (FA). The flow-through was pooled in a 96-wells plate which was centrifuged to dryness in a vacuum centrifuge.

For sample desalting, peptides were resuspended in 100 μL of 0.1% FA and transferred to a 96 wells MACROSPIN C18 plate (Targa, Nest Group, MA, USA) which had previously been activated with 100 μL of 70% ACN, 0.1% FA followed conditioning with 100 μL 0.1% FA. To transfer the solutions through the MACROSPIN C18 plate, the plates were centrifuged at 2000 g for two minutes. After capturing the peptides on the C18 beads the plate was washed with 100 μL of 0.1% FA followed by eluting the peptides with 100 μL 40% ACN, 0.1% FA and 100 μL 70% ACN, 0.1% FA. The captured eluents were dried down in a vacuum centrifuge and stored at −20° C. until analysis.

LC-MS/MS analysis: To validate the biomarker candidates the prepared CSF samples were analyzed on an Orbitrap Q Exactive mass spectrometer (Thermo Scientific, Bremen, Germany). First, the tryptic digests were resuspended in 20 μL resuspension buffer (5% ACN, 5% FA) and placed into a nanoflow HPLC pump module LC autosampler (Eksigent/Sciex, Framingham, MA, USA) where 4 μL of the sample was loaded onto a PicoChip column (150 μm×10 cm Acquity BEH C18 1.7 μm 130 Å, New Objective, Woburn, MA) which was kept at 50° C. The peptides were eluted off the PicoChip column using 2% of solvent B (0.1% FA in ACN) in solvent A (0.1% FA), which was increased from 2 to 30% in a 40 min ramp gradient and back to 35% on a 5 min ramp gradient with a flow rate of 1000 nL/min. The Orbitrap settings were the following: positive DDA top 12 mode. MS1 scan settings: m/z range: 375-1400, resolution 70000 @m/z 200, AGC target 3e6, max IT 60 ms. MS scan settings: resolution 17500 @m/z 200, AGC target 1e5, max IT 100 ms, isolation window m/z 1.6, NCE 27, underfill ratio 1% (intensity threshold 1e4), charge state exclusion unassigned, 1, >6, peptide match preferred, exclude isotopes on, dynamic exclusion 40 s.

Statistical analysis: Given our goal of mining and (re-)analyzing existing data using a standard and systematic data processing pipeline, all methods described above were also applied to this set of CSF samples, apart from Z-scoring the data. First, a two-sided Fisher's exact test with a Benjamini-Hochberg correction was used to determine if a protein was only identified in the AD or control group. Next, proteins with valid values less than 50% were removed from the dataset followed by analysis with a Mann-Whitney U-test and a Benjamini-Hochberg Correction. Furthermore, the effect of age and gender on the biomarker candidates were assessed using logistic regression analysis. Logistic regression analysis was carried out in R; for each detected biomarker candidate, the effect of age, sex and interactions between age or sex and the biomarker candidate were assessed by including their separate interaction terms. Last, specificity and sensitivity of the validation cohort were assessed with a Receiver-operating characteristic (ROC) analysis.

A panel of proteins may be better in differentiating (greater sensitivity and specificity) in AD from controls when compared to a single protein candidate. The predictive ability of the biomarker panel was assessed using ROC analysis using R. The panel was modelled with the meta-analysis data and tested on the validation cohort, as well as on data of two recent large scale AD CSF publications (14, 15).

### REFERENCES

1. G. M. Sancesario, S. Bernardini, Diagnosis of neurodegenerative dementia: where do we stand, now?, Annals of Translational Medicine 6, 12 (2018).
2. S. Karantzoulis, J. E. Galvin, Distinguishing Alzheimer's disease from other major forms of dementia, Expert Rev Neurother 11, 1579-1591 (2011).
3. K. Blennow, B. Dubois, A. M. Fagan, P. Lewczuk, M. J. de Leon, H. Hampel, Clinical utility of cerebrospinal fluid biomarkers in the diagnosis of early Alzheimer's disease, Alzheimer's & Dementia 11, 58-69 (2015).
4. C. Teunissen, C. Verheul, E. Willemse, in Handbook of clinical neurology, (Elsevier, 2018), vol. 146, pp. 3-20.
5. B. Aslam, M. Basit, M. A. Nisar, M. Khurshid, M. H. Rasool, Proteomics: technologies and their applications, Journal of chromatographic science 55, 182-196 (2017).
6. B. Carlyle, B. Trombetta, S. Arnold, Proteomic Approaches for the Discovery of Biofluid Biomarkers of Neurodegenerative Dementias, Proteomes 6, 32 (2018).
7. R. Cunningham, D. Ma, L. Li, Mass Spectrometry-based Proteomics and Peptidomics for Systems Biology and Biomarker Discovery, Front Biol (Beijing) 7, 313-335 (2012).
8. H. Mischak, J. P. A. Ioannidis, A. Argiles, T. K. Attwood, E. Bongcam-Rudloff, M. Broenstrup, A. Charonis, G. P. Chrousos, C. Delles, A. Dominiczak, T. Dylag, J. Ehrich, J. Egido, P. Findeisen, J. Jankowski, R. W. Johnson, B. A. Julien, T. Lankisch, H. Y. Leung, D. Maahs, F. Magni, M. P. Manns, E. Manolis, G. Mayer, G. Navis, J. Novak, A. Ortiz, F. Persson, K. Peter, H. H. Riese, P. Rossing, N. Sattar, G. Spasovski, V. Thongboonkerd, R. Vanholder, J. P. Schanstra, A. Vlahou, Implementation of proteomic biomarkers: making it work: IMPLEMENTATION OF PROTEOMIC BIOMARKERS, European Journal of Clinical Investigation 42, 1027-1036 (2012).
9. D. Orton, A. Doucette, Proteomic Workflows for Biomarker Identification Using Mass Spectrometry—Technical and Statistical Considerations during Initial Discovery, Proteomes 1, 109-127 (2013).
10. F. Abdi, J. F. Quinn, J. Jankovic, M. McIntosh, J. B. Leverenz, E. Peskind, R. Nixon, J. Nutt, K. Chung, C. Zabetian, Detection of biomarkers with a multiplex quantitative proteomic platform in cerebrospinal fluid of patients with neurodegenerative disorders, Journal of Alzheimer's Disease 9, 293-348 (2006).
11. A. Brinkmalm, E. Portelius, A. Öhrfelt, G. Brinkmalm, U. Andreasson, J. Gobom, K. Blennow, H. Zetterberg, Explorative and targeted neuroproteomics in Alzheimer's disease, Biochimica et Biophysica Acta (BBA)—Proteins and Proteomics 1854, 769-778 (2015).
12. J. M. Ringman, H. Schulman, C. Becker, T. Jones, Y. Bai, F. Immermann, G. Cole, S. Sokolow, K. Gylys, D. H. Geschwind, Proteomic changes in cerebrospinal fluid of presymptomatic and affected persons carrying familial Alzheimer disease mutations, Archives of neurology 69, 96-104 (2012).
13. G. N. Yin, H. W. Lee, J. Y. Cho, K. Suk, Neuronal pentraxin receptor in cerebrospinal fluid as a potential biomarker for neurodegenerative diseases, Brain research 1265, 158-170 (2009).
14. J. M. Bader, P. E. Geyer, J. B. Müller, M. T. Strauss, M. Koch, F. Leypoldt, P. Koertvelyessy, D. Bittner, C. G. Schipke, E. I. Incesoy, O. Peters, N. Deigendesch, M. Simons, M. K. Jensen, H. Zetterberg, M. Mann, Proteome profiling in cerebrospinal fluid reveals novel biomarkers of Alzheimer's disease, Mol Syst Biol 16 (2020), doi:10.15252/msb.20199356.
15. E. C. B. Johnson, E. B. Dammer, D. M. Duong, L. Ping, M. Zhou, L. Yin, L. A. Higginbotham, A. Guajardo, B. White, J. C. Troncoso, M. Thambisetty, T. J. Montine, E. B. Lee, J. Q. Trojanowski, T. G. Beach, E. M. Reiman, V. Haroutunian, M. Wang, E. Schadt, B. Zhang, D. W. Dickson, N. Ertekin-Taner, T. E. Golde, V. A. Petyuk, P. L. De Jager, D. A. Bennett, T. S. Wingo, S. Rangaraju, I. Hajjar, J. M. Shulman, J. J. Lah, A. I. Levey, N. T. Seyfried, Large-scale proteomic analysis of Alzheimer's disease brain and cerebrospinal fluid reveals early changes in energy metabolism associated with microglia and astrocyte activation, Nat Med (2020), doi:10.1038/s41591-020-0815-6.
16. L. Dayon, O. Cominetti, J. Wojcik, A. Núñez Galindo, A. Oikonomidi, H. Henry, E. Migliavacca, M. Kussmann, G. Bowman, J. Popp, Proteomes of paired human cerebrospinal fluid and plasma: Relation to blood-brain barrier permeability in older adults, Journal of proteome research (2019).
17. G. Sathe, C. H. Na, S. Renuse, A. K. Madugundu, M. Albert, A. Moghekar, A. Pandey, Quantitative Proteomic Profiling of Cerebrospinal Fluid to Identify Candidate Biomarkers for Alzheimer's Disease, PROTEOMICS—Clinical Applications, 1800105 (2018).
18. P. E. Khoonsari, A. Häggmark, M. Lönnberg, M. Mikus, L. Kilander, L. Lannfelt, J. Bergquist, M. Ingelsson, P. Nilsson, K. Kultima, Analysis of the cerebrospinal fluid proteome in Alzheimer's disease, PloS one 11, e0150672 (2016).
19. A. Lleó, R. Núñez-Llaves, D. Alcolea, C. Chiva, D. Balateu-Panos, M. Colom-Cadena, G. Gomez-Giro, L. Muñoz, M. Querol-Vilaseca, J. Pegueroles, Changes in Synaptic Proteins Precede Neurodegeneration Markers in Preclinical Alzheimer's Disease Cerebrospinal Fluid, Molecular & Cellular Proteomics 18, 546-560 (2019).
20. C. Barucker, A. Sommer, G. Beckmann, M. Eravci, A. Harmeier, C. G. Schipke, D. Brockschnieder, T. Dyrks, V. Althoff, P. E. Fraser, Alzheimer amyloid peptide Aβ 42 regulates gene expression of transcription and growth factors, Journal of Alzheimer's disease 44, 613-624 (2015).
21. J. Wang, R. Cunningham, H. Zetterberg, S. Asthana, C. Carlsson, O. Okonkwo, L. Li, Label-free quantitative comparison of cerebrospinal fluid glycoproteins and endogenous peptides in subjects with Alzheimer's disease, mild cognitive impairment, and healthy individuals, PROTEOMICS—Clinical Applications 10, 1225-1241 (2016).
22. W. E. Heywood, D. Galimberti, E. Bliss, E. Sirka, R. W. Paterson, N. K. Magdalinou, M. Carecchio, E. Reid, A. Heslegrave, C. Fenoglio, Identification of novel CSF biomarkers for neurodegeneration and their validation by a high-throughput multiplexed targeted proteomic assay, Molecular neurodegeneration 10, 64 (2015).
23. P. Emami Khoonsari, G. Shevchenko, S. Herman, S. Musunuri, J. Remnestål, R. Brundin, M. Degerman Gunnarsson, L. Kilander, H. Zetterberg, P. Nilsson, Chitinase-3-like protein 1 (CH3L1) and Neurosecretory protein VGF (VGF) as two novel CSF biomarker candidates for improved diagnostics in Alzheimer's disease, (2017).
24. O. F. Laterza, V. R. Modur, D. L. Crimmins, J. V. Olander, Y. Landt, J. M. Lee, J. H. Ladenson, Identification of novel brain biomarkers, Clinical chemistry 52, 1713-1721 (2006).
25. H. Selle, J. Lamerz, K. Buerger, A. Dessauer, K. Hager, H. Hampel, J. Karl, M. Kellmann, L. Lannfelt, J. Louhija, Identification of novel biomarker candidates by differential peptidomics analysis of cerebrospinal fluid in Alzheimer's disease, Combinatorial chemistry & high throughput screening 8, 801-806 (2005).
26. D. Szklarczyk, A. L. Gable, D. Lyon, A. Junge, S. Wyder, J. Huerta-Cepas, M. Simonovic, N. T. Doncheva, J. H. Morris, P. Bork, STRING v11: protein-protein association networks with increased coverage, supporting functional discovery in genome-wide experimental datasets, Nucleic acids research 47, D607-D613 (2019).
27. Y. An, V. R. Varma, S. Varma, R. Casanova, E. Dammer, O. Pletnikova, C. W. Chia, J. M. Egan, L. Ferrucci, J. Troncoso, A. I. Levey, J. Lah, N. T. Seyfried, C. Legido-Quigley, R. O'Brien, M. Thambisetty, Evidence for brain glucose dysregulation in Alzheimer's disease, Alzheimers Dement 14, 318-329 (2018).
28. E. Trushina, T. Dutta, X. M. T. Persson, M. M. Mielke, R. C. Petersen, Identification of altered metabolic pathways in plasma and CSF in mild cognitive impairment and Alzheimer's disease using metabolomics, PloS one 8, e63644 (2013).
29. M. Perluigi, R. Sultana, G. Cenini, F. Di Domenico, M. Memo, W. M. Pierce, R. Coccia, D. A. Butterfield, Redox proteomics identification of 4-hydroxynonenal-modified brain proteins in Alzheimer's disease: role of lipid peroxidation in Alzheimer's disease pathogenesis, PROTEOMICS—Clinical Applications 3, 682-693 (2009).
30. A. Tramutola, C. Lanzillotta, M. Perluigi, D. A. Butterfield, Oxidative stress, protein modification and Alzheimer disease, Brain Research Bulletin 133, 88-96 (2017).
31. P. H. Reddy, R. Tripathi, Q. Troung, K. Tirumala, T. P. Reddy, V. Anekonda, U. P. Shirendeb, M. J. Calkins, A. P. Reddy, P. Mao, Abnormal mitochondrial dynamics and synaptic degeneration as early events in Alzheimer's disease: implications to mitochondria-targeted antioxidant therapeutics, Biochimica et Biophysica Acta (BBA)-Molecular Basis of Disease 1822, 639-649 (2012).
32. K. Schmitt, A. Grimm, A. Kazmierczak, J. B. Strosznajder, J. Götz, A. Eckert, Insights into mitochondrial dysfunction: aging, amyloid-β, and tau—a deleterious trio, Antioxidants & redox signaling 16, 1456-1466 (2012).
33. J. E. Selfridge, E. Lezi, J. Lu, R. H. Swerdlow, Role of mitochondrial homeostasis and dynamics in Alzheimer's disease, Neurobiology of disease 51, 3-12 (2013).
34. J. Yao, J. R. Rettberg, L. P. Klosinski, E. Cadenas, R. D. Brinton, Shift in brain metabolism in late onset Alzheimer's disease: implications for biomarkers and therapeutic interventions, Molecular aspects of medicine 32, 247-257 (2011).
35. M. Zhou, R. U. Haque, E. B. Dammer, D. M. Duong, L. Ping, E. C. B. Johnson, J. J. Lah, A. I. Levey, N. T. Seyfried, Targeted mass spectrometry to quantify brain-derived cerebrospinal fluid biomarkers in Alzheimer's disease, Clinical Proteomics 17, 19 (2020).
36. J. A. Vizcaino, A. Csordas, N. Del-Toro, J. A. Dianes, J. Griss, I. Lavidas, G. Mayer, Y. Perez-Riverol, F. Reisinger, T. Ternent, 2016 update of the PRIDE database and its related tools, Nucleic acids research 44, D447-D456 (2015).
37. J. Cox, M. Mann, MaxQuant enables high peptide identification rates, individualized ppb-range mass accuracies and proteome-wide protein quantification, Nature biotechnology 26, 1367 (2008).
38. D. S. Spellman, K. R. Wildsmith, L. A. Honigberg, M. Tuefferd, D. Baker, N. Raghavan, A. C. Nairn, P. Croteau, M. Schirm, R. Allard, Development and evaluation of a multiplexed mass spectrometry based assay for measuring candidate peptide biomarkers in Alzheimer's Disease Neuroimaging Initiative (ADNI) CSF, PROTEOMICS—Clinical Applications 9, 715-731 (2015).
39. S. Tyanova, T. Temu, P. Sinitcyn, A. Carlson, M. Y. Hein, T. Geiger, M. Mann, J. Cox, The Perseus computational platform for comprehensive analysis of (prote) omics data, Nature methods 13, 731 (2016).
40. Michael Waskom, Olga Botvinnik, Drew O'Kane, Paul Hobson, Saulius Lukauskas, David C Gemperline, Tom Augspurger, Yaroslav Halchenko, John B. Cole, Jordi Warmenhoven, Julian de Ruiter, Cameron Pye, Stephan Hoyer, Jake Vanderplas, Santi Villalba, Gero Kunter, Eric Quintero, Pete Bachant, Marcel Martin, Kyle Meyer, Alistair Miles, Yoav Ram, Tal Yarkoni, Mike Lee Williams, Constantine Evans, Clark Fitzgerald, Brian, Chris Fonnesbeck, Antony Lee, Adel Qalieh, mwaskom/seaborn: v0.8.1 (September 2017) (Zenodo, 2017; https://zenodo.org/record/883859#.XsQBWmhKiUk).
41. RStudio-Team., RStudio: Integrated Development for R. (2015).
42. Y. Benjamini, Y. Hochberg, Controlling the false discovery rate: a practical and powerful approach to multiple testing, Journal of the Royal statistical society: series B (Methodological) 57, 289-300 (1995).
43. K. Blighe, EnhancedVolcano: Publication-ready volcano plots with enhanced colouring and labeling. (https://github.com/kevinblighe, 2018).
44. M. Ashburner, C. A. Ball, J. A. Blake, D. Botstein, H. Butler, J. M. Cherry, A. P. Davis, K. Dolinski, S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin, G. Sherlock, Gene Ontology: tool for the unification of biology, Nature Genetics 25, 25-29 (2000).
45. The Gene Ontology Consortium, The Gene Ontology Resource: 20 years and still GOing strong, Nucleic Acids Res 47, D330-D338 (2019).
46. G. Bindea, B. Mlecnik, H. Hackl, P. Charoentong, M. Tosolini, A. Kirilovsky, W. H. Fridman, F. Pagès, Z. Trajanoski, J. Galon, ClueGO: a Cytoscape plug-in to decipher functionally grouped gene ontology and pathway annotation networks, Bioinformatics 25, 1091-1093 (2009).
47. O. Hansson, H. Zetterberg, P. Buchhave, E. Londos, K. Blennow, L. Minthon, Association between CSF biomarkers and incipient Alzheimer's disease in patients with mild cognitive impairment: a follow-up study, Lancet Neurol 5, 228-234 (2006).
48. T. B. Bennike, H. Steen, in Serum/Plasma Proteomics, (Springer, 2017), pp. 395-402.
49. T. B. Bennike, M. D. Bellin, Y. Xuan, A. Stensballe, F. T. Møoller, G. J. Beilman, O. Levy, Z. Cruz-Monserrate, V. Andersen, J. Steen, A cost-effective high-throughput plasma and serum proteomics workflow enables mapping of the molecular impact of total pancreatectomy with islet autotransplantation, Journal of proteome research 17, 1983-1992 (2018).
50. S. T. Berger, S. Ahmed, J. Muntel, N. C. Polo, R. Bachur, A. Kentsis, J. Steen, H. Steen, MStern blotting—high throughput polyvinylidene fluoride (PVDF) membrane-based proteomic sample preparation for 96-well plates, Molecular & Cellular Proteomics 14, 2814-2823 (2015).

### Example 2

Provided herein are three cerebrospinal fluid protein-based biomarker panels that can be used in the diagnosis of Alzheimer's Disease (AD) in patients. These biomarker panels were derived from a large-scale meta-analysis of published LC-MS based proteomics studies for Alzheimer's Disease cerebrospinal fluid (CSF) biomarker studies. This meta cohort included 73 AD cases and 77 controls from 7 independent studies (6 previously published studies and 1 in-house study) in 5 different countries. Statistical analysis of the meta cohort revealed 37 biomarker candidates which were further validated in three independent validation cohorts totaling 514 samples. The validation cohorts did result in highly significant biomarker candidates, which were used to develop three biomarker panels that are proposed herein.

Panel one: Panel one consists of the proteins Pyruvate Kinase (PKM), Fructose-bisphosphate aldolase A (ALDOA) and L-lactate dehydrogenase B chain (LDHB), which are all key enzymes in metabolic pathways. For validation cohort one (10 AD samples, 10 control samples) an area under the receiver operating characteristic curve (AUROC) of 1.00 was reached. For validation cohort two (150 AD, 147 control) an AUROC of 0.88 was reached and for validation cohort three (88AD, 109 control) and AUROC of 0.83 was reached.

Panel two: Panel two consists of the proteins PKM, ALDOA, LDHB and 14-3-3 protein zeta/delta (YWHAZ). An AUROC of panel two in validation cohort one was not available, because here YWHAZ was not identified. Validation cohort two and three scored an AUROC of 0.92 and 0.86, respectively.

Panel three: Panel three consists of the PKM, ALDOA, LDHB and Apolipoprotein E isoform 4 (APOE-4). Validation cohort one reached an AUROC of 1.00, validation cohort two an AUROC of 0.78 and validation cohort three an AUROC of 0.82.

Without wishing to be bound by theory, the inventors understand that PKM, ALDOA and LDHB represent brain glucose homeostasis pathology in the AD brain, whereas YWHAZ and APOE-4 are other unique characteristics of AD pathology in CSF. In conclusion, the inventors envision that these biomarker panels as effective tools for the in vivo diagnosis of AD.

1. A method for treating Alzheimer's disease in a subject, the method comprising:
(i) receiving results of a protein assay relating to the expression of a panel of proteins comprising pyruvate kinase (PKM), fructose bisphosphate aldolase A (ALDOA), and lactate dehydrogenase B chain (LDHB) in a biological sample obtained from a subject,
(ii) determining that the expression of at least one of the members of the panel of proteins is increased in the biological sample compared to the expression of the at least one member in a reference sample, and
(iii) administering a treatment for Alzheimer's disease to the subject, thereby treating Alzheimer's disease in the subject.

2. (canceled)

3. The method of claim 1, wherein the biological sample is cerebrospinal fluid (CSF), whole blood, plasma, serum, or urine.

4. The method of claim 1, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

5. The method of claim 1, further comprising a step of obtaining the biological sample from the subject.

6. The method of claim 1, further comprising a step of selecting the subject based on the presence or expression level of apolipoprotein E isoform 4 (apoE4) and/or 14-3-3 protein zeta/delta (YWHAZ).

7. The method of claim 1, wherein the treatment for Alzheimer's disease comprises a cholinesterase inhibitor, intravenous immunoglobulin (IVIG), aducanumab, or memantine.

8. The method of claim 7, wherein the cholinesterase inhibitor comprises donepezil, galantamine, or rivastigmine.

9. The method of claim 1, further comprising repeating steps (i) and (ii) at a later time point after treatment has been initiated to monitor efficacy of the treatment.

10. A method for treating Alzheimer's disease in a subject, the method comprising:
(i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and 14-3-3 protein zeta/delta (YWHAZ),
(ii) determining that expression of at least one of the members of the panel of proteins is increased in the biological sample as compared to the expression level of the at least one member in a reference sample, and
(iii) administering a treatment for Alzheimer's disease to the subject, thereby treating Alzheimer's disease in the subject.

11-18. (canceled)

19. A method for treating Alzheimer's disease in a subject, the method comprising:
(i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), and apolipoprotein E isoform 4 (apoE4),
(ii) determining that the expression of at least one of the members of the panel of proteins is increased in the biological sample as compared to the expression of the at least one member in a reference sample, and
(iii) administering a treatment for Alzheimer's disease to the subject, thereby treating Alzheimer's disease in the subject.

20-26. (canceled)

27. A method for treating Alzheimer's disease in a subject, the method comprising:
(i) receiving results of a protein assay relating to the expression of each member of a panel of proteins in a biological sample obtained from a subject, wherein the panel of proteins comprises pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4),
(ii) determining that the expression of at least one of the members of the panel of proteins is increased in the biological sample as compared to the expression of the at least one member in a reference sample, and
(iii) administering a treatment for Alzheimer's disease to the subject, thereby treating Alzheimer's disease in the subject.

28. (canceled)

29. The method of claim 27, wherein the biological sample is cerebrospinal fluid (CSF), whole blood, plasma, serum, or urine.

30. The method of claim 27, wherein the protein assay comprises an enzyme-linked immunosorbent (ELISA) assay, mass spectrometry, western blot, 2-D gel electrophoresis, microarray-based method, proximity extension assays, slow-offrate-modified-aptamer reagent (SOMAmer), or a nanoscale needle biosensor assay.

31. The method claim 27, further comprising a step of obtaining the biological sample from the subject.

32. The method of claim 27, wherein the treatment for Alzheimer's disease comprises a cholinesterase inhibitor, intravenous immunoglobulin (IVIG), aducanumab, or memantine.

33. The method of claim 32, wherein the cholinesterase inhibitor comprises donepezil, galantamine, or rivastigmine.

34. The method of claim 27, further comprising repeating steps (i) and (ii) at a later time point after treatment has been initiated to monitor efficacy of the treatment.

35. A method for treating Alzheimer's disease in a subject, the method comprising:
(i) receiving results of a protein assay relating to the expression of each member of a panel of proteins in a biological sample obtained from a subject, wherein the panel of proteins comprises at least two of the members of, or consists essentially of, pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4),
(ii) determining that the expression of at least one of the members of the panel of proteins is increased in the biological sample as compared to the expression of the at least one member in a reference sample; and
(iii) administering a treatment for Alzheimer's disease to the subject, wherein the treatment reduces or alleviates one or more symptoms of Alzheimer's disease in the subject, thereby treating Alzheimer's disease in the subject.

36. (canceled)

37. The method of claim 36, wherein the subject is selected for participation in a clinical trial for an Alzheimer's disease agent.

38. The method of claim 37, wherein the Alzheimer's disease agent is an early-stage intervention for Alzheimer's disease.

39. The method of claim 1, wherein the treatment reduces or alleviates one or more symptoms of Alzheimer's disease in the subject, wherein the one or more symptoms of Alzheimer's disease comprise: impaired cognitive function, worsening cognitive function, paranoia, episodes of forgetting, confusion, disorientation, agitation, irritability, depression, hallucinations, difficulty concentrating, impaired mathematical reasoning, impaired ability to form new memories, anger or combinations thereof.

40. The method of claim 35, wherein the panel of proteins comprises:
(a) pyruvate kinase (PKM), fructose bisphosphate aldolase A (ALDOA), and lactate dehydrogenase B chain (LDHB);
(b) pyruvate kinase (PKM), fructose bisphosphate aldolase A (ALDOA), lactate dehydrogenase B chain (LDHB), and 14-3-3 protein zeta/delta (YWHAZ);
(c) pyruvate kinase (PKM), fructose bisphosphate aldolase A (ALDOA), lactate dehydrogenase B chain (LDHB), and apolipoprotein E isoform 4 (apoE4); or
(d) comprises pyruvate kinase (PKM), fructose bisphosphate aldolase A (ALDOA), lactate dehydrogenase B chain (LDHB), 14-3-3 protein zeta/delta (YWHAZ) and apolipoprotein E isoform 4 (apoE4).


SEQUENCE LISTING (5 sequences)

SEQ ID NO: 1 - protein, 54 aa, Homo sapiens
LNFSHGTHEYHAETIKTATESFASDPILYRPVAVALDTKGPEIRSVETLKEMIK

SEQ ID NO: 2 - protein, 46 aa, Homo sapiens
LQSIGTENTEENRRADDGRPFPQVIKIGEHTPSALAIMENANVLAR

SEQ ID NO: 3 - protein, 34 aa, Homo sapiens
IVADKDYSVTANSKIVVVTAGVRLKDDEVAQLKK

SEQ ID NO: 4 - protein, 31 aa, Homo sapiens
NLLSVAYKMDKNELVQKSVTEQGAELSNEER

SEQ ID NO: 5 - protein, 48 aa, Homo sapiens
LGADMEDVCGRLGADMEDVRLAVYQAGARCLAVYQAGARLGPLVEQGR